SEQUENCE LISTING (93 sequences)

SEQ ID NO: 1 - protein, 8 aa, Homo sapiens
VVDMRRYF

SEQ ID NO: 2 - protein, 132 aa, Homo sapiens
MVTAGHACTKKYTPEQVAMATVTALHRTVPAAVPGICFLSGGMSEEDATLNLNAINLCPLPKPWKLSFSYGRALQASALAAWGGKAANKEATQEAFMKRAMANCQAAKGQYVHTGSSGAASTQSLFTACYTY

SEQ ID NO: 3 - protein, 5 aa, Homo sapiens
IIIGS

SEQ ID NO: 4 - protein, 5 aa, Homo sapiens
SLFLM

SEQ ID NO: 5 - protein, 128 aa, Homo sapiens
LVALPATPLTPAFPPPSFDQRSAETLEVRKLDTHPDRTGVLRGVAGGFGLDEFPPKCRRSQARAQKQRMGKSMQHSVYLILTRGNHKHRKPQLDMDYRVLEKSVFHGVCLHLLTSQVLKQKTKAMILM

SEQ ID NO: 6 - protein, 25 aa, Homo sapiens
RKINPLIKLINHSFIDLPTPSNISA

SEQ ID NO: 7 - protein, 19 aa, Homo sapiens
SVRGSVVGSTLTPLACPDP

SEQ ID NO: 8 - protein, 15 aa, Homo sapiens
QLCWLDWLPCSWPRT

SEQ ID NO: 9 - protein, 5 aa, Homo sapiens
SFGSL

SEQ ID NO: 10 - protein, 7 aa, Homo sapiens
RKNKLRQ

SEQ ID NO: 11 - protein, 21 aa, Homo sapiens
LREKMTGYIQSDMAKSLKVLM

SEQ ID NO: 12 - protein, 132 aa, Homo sapiens
  misc_feature (124)..(124): “Xaa” may be any amino acid
RVTIRKSKNILFVITKPDVYKSPASDTYIVFGEAKIEDLSQQAQLAAAEKFKVQGEAVSNIQENTQTPTVQEESEEEEVDETGVEVKDIELVMSQANVSRAKAVRALKNNSNDIVNAIMELTMXPYGSNFFW

SEQ ID NO: 13 - protein, 23 aa, Homo sapiens
KGKNDWLYSVRYGKKSQGVNVND

SEQ ID NO: 14 - protein, 66 aa, Homo sapiens
EHMVITDRIENIDHLGFFIYRLCHDKETYKLQRRETIKGIQKREASNCFAIRHFENKFAVETLICS

SEQ ID NO: 15 - protein, 180 aa, Homo sapiens
GGEEPAEEDSEDWCVPCSDEEVELPADGQPWMPPPSEIQRLYELLAAHGTLELQAEILPRRPPTPEAQSEEERSDEEPEAKEEEEEKPHMPTEFDFDDEPVTPKDSLIDRRRTPGSSARSQKREARLDKVLSDMKRHKKLEEQILRTGRDLFSLDSEDPSPASPPLRSSGSSLFPRQRKY

SEQ ID NO: 16 - protein, 19 aa, Homo sapiens
RTVSSINGVGKTGYPYAKE

SEQ ID NO: 17 - protein, 19 aa, Homo sapiens
NLGLLFILATSSLAVYSIL

SEQ ID NO: 18 - protein, 30 aa, Homo sapiens
KSMKNNPVIVFATKGKQCKMTYSISSCSNY

SEQ ID NO: 19 - protein, 163 aa, Homo sapiens [sequence omitted]

SEQ ID NO: 20 - protein, 105 aa, Homo sapiens
KNALAHFLPQGTPTPLIPILVIIETISLLIQPIALAVRLTANITAGHLLMHLIGSATLAISTINLPSTLIIFTILILLTILEIAVALIQAYVFTLLVSLYLHDNT

SEQ ID NO: 21 - protein, 117 aa, Homo sapiens
ARPILRIISLLRNLKHRHYPPACNYSNSLHRLCPPVRPNIILRGHSNYKLTIRHPIHWDRPSSMNLRRLLSRQSHPHTILYLSLHLALHYCSPSSTPPPILARNGIKQPPRNHLPFR

SEQ ID NO: 22 - protein, 24 aa, Homo sapiens
EPSGSCANRHQYQLCELGCECTRA

SEQ ID NO: 23 - protein, 520 aa, Homo sapiens
PTDSTMLKKFDKKDEESGGGSNPFQHLEKSAVLQEARVFNETPINPRKCAHILTKILYLINQGEHLGTTEATEAFFAMTKLFQSNDPTLRRMCYLTIKEMSCIAEDVIIVTSSLTKDMTGKEDNYRGPAVRALCQITDSTMLQAIERYMKQAIVDKVPSVSSSALVSSLHLLKCSFDVVKRWVNEAQEAASSDNIMVQYHALGLLYHVRKNDRLAVNKMISKVTRHGLKSPFAYCMMIRVASKQLEEEDGSRDSPLFDFIESCLRNKHEMVVYEAASAIVNLPGCSAKELAPAVSVLQLFCSSPKAALRYAAVRTLNKVAMKHPSAVTACNLDLENLVTDSNRSIATLAITTLLKTGSESSIDRLMKQISSFMSEISDEFKVVVVQAISALCQKYPRKHAVLMNFLFTMLREEGGFEYKRAIVDCIISIIEENSESKETGLSHLCEFIEDCEFTVLATRILHLLGQEGPKTTNPSKYIRFIYNRVVLEHEEVRAGAVSALAKFGAQNEEMLPSILVLLKR

SEQ ID NO: 24 - protein, 21 aa, Homo sapiens
PQTHSTLLPDNLSQTIYPNKV

SEQ ID NO: 25 - protein, 46 aa, Homo sapiens
HNKQTWSILGLLHALPEPVPAAGGSPGERLWPLQLLRECARQTRQI

SEQ ID NO: 26 - protein, 34 aa, Homo sapiens
NTKICQAWWRTPIIPATGEAEAGEPLEPRRQRLQ

SEQ ID NO: 27 - protein, 180 aa, Homo sapiens
GLLKVVFVVFASLCAWYSGYLLAELIPDAPLSSAAYSIRSIGERPVLKGECRAWGRRLPSWLVCRGRSGGFCPSWRLGGPDGFISGRRRREAFCSYSFRSHAIPRHVPLLTPCETRTVWLLVTSTNLQPDLELCSFRPDLGFLPWEAQEGPGSEIPNHFKVSVGLKSCWKIFCKVLGSKS

SEQ ID NO: 28 - protein, 34 aa, Homo sapiens
KKKSTWVQWLTPVILAFGETKVGGSLEPGRSRLQ

SEQ ID NO: 29 - protein, 7 aa, Homo sapiens
RSCHDLN

SEQ ID NO: 30 - protein, 237 aa, Homo sapiens
YAAEICIALNFLHERGIIYRDLKLDNVLLDADGHIKLTDYGMCKEGLGPGDTTSTFCGTPNYIAPEILRGEEYGFSVDWWALGVLMFEMMAGRSPFDIITDNPDMNTEDYLFQVILEKPIRIPRFLSVKASHVLKGFLNKDPKERLGCRPQTGFSDIKSHAFFRSIDWDLLEKKQALPPFQPQITDDYGLDNFDTQFTSEPVQLTPDDEDAIKRIDQSEFEGFEYINPLLLSTEESV

SEQ ID NO: 31 - protein, 54 aa, Homo sapiens
FWILPSISRPLRARHFRAGQNLCRSRCGFAGDQGGDEKGRGGAPAQERGCGAPG

SEQ ID NO: 32 - protein, 26 aa, Homo sapiens
KKKKYLGTVAHTCNPSIWGDQGGRIT

SEQ ID NO: 33 - DNA, 1145 bp, Homo sapiens
gttgtagatatgcggcgttatttctgagggctctgttctgttccattgatctatatctctgtttcggtaccagtaccatgctgttactgtagccttgtagtatagtttgaaatcaggtagcgtgatgcctccagctttgttctttcggcttaggattgacttggcaatgcgggctcttttttggttccatatgaactttaaagtagttttttccaattctgtgaagaaagtgattggtagcttgatggggatggcattgaatctataaattaccttaggcagtatggccattttcacgatattgattcttcctacccatgagcatggaatgttcttccatttctttgtatcctcttttatttcattgagcagtggtttgtagttctccttgatgaggtccttcacaaaaactaacaaacagaaaggacgtgacagcccagagtttttaaagctgacacttgagttgggtttttctctttctttaccactgtaaaaaaataatgaatgagaattgggtgctgggttttgttttgttgttttagtggtggcagggaagctggggatgcgggtgattaggctgagtattacagaggtagcaggagtcccatagcaccaagttccatcttcttgttacccattccagaggccccaaacctacctagaggctttgggatcaggaggcttgagcttggattctggctctgccacctccttacctagaacccctttgtaagtcactgagtttgtgagtctcaacgtcgtcattacaaaagtacagaaacaccaaccttctctttcatggtgggtagtgtggtagtactggactctggatgagttatactttaaaagctatcaactgtatatcatgtaatcctgcccctacaggactgattcactaacaagataactgtttaaaagaaagaaaaagagagcggggcgcagtggctcacacctgtaatcccagcactttgggaggctgaggcgggtggatcatttgaggtcaggagtttgaggccagcctggccaacatagtgaaaccctgtctctaccagaaatacaaaaaaattagccgggtgtggtagtgcatgcctataatcccagctacttgggaggctgaggcaggagaatcacttgaacccgggagg

SEQ ID NO: 34 - DNA, 929 bp, Homo sapiens
aacactgccagtatgttactgagaaggtcctggctgctgtctacaaggccctgaatgaccatcatgtttacctggagggcaccctgctaaagcccaacatggtgactgctggacatgcctgcaccaagaagtatactccagaacaagtagctatggccaccgtaacagctctccaccgtactgttcctgcagctgttcctggcatctgctttttgtctggtggcatgagtgaagaggatgccactctcaacctcaatgctatcaacctttgccctctaccaaagccctggaaactaagtttctcttatggacgggccctgcaggccagtgcactggctgcctggggtggcaaggctgcaaacaaggaggcaacccaggaggcttttatgaagcgggccatggctaactgccaggcggccaaaggacagtatgttcacacgggttcttctggggctgcttccacccagtcgctcttcacagcctgctatacctactagggtccaatgcccgccagcctagctccagtgcttctagtaggagggctgaaagggagcaacttttcctctaatcctggaaattcgacacaattagatttgaactgctggaaatacaacacatgttaaatcttaagtacaagggggaaaaaataaatcagttattgaaacataaaaatgaataccaaggacctgatcaaatttcacacagcagtttccttgcaacactttcagctccccatgctccagaatacccacccaagaaaataataggctttaaaacaatatcggctcctcatccaaagaacaactgctgattgaaacacctcattagctgagtgtagagaagtgcatcttatgaaacagtcttagcagtggtaggttgggaaggagatagctgcaaccaaaaaagaaataaatattctataaaccttc

SEQ ID NO: 35 - DNA, 479 bp, Homo sapiens
cataatcataggtagctaggttataaactatttaaagacaagatcacgtgataagcttataatcttctcataattccccttacttagcattgtgttagacatactaataggtgcacagtgaaatacttattgttgattgtttaaaaataaagttttagaaaaccttttcaaaagtcagagtttaggccaggggcacaggctgacacctataatcccagcactttgggaggccagggcgggcagatcacttgggtcaagagttcaaggccagcctggccaacatggcaaaaccccatctctactaaataaaatacaaaaattatccaggcatggtggtgcatgcctgtaatcccagctacttggaggctgaggcatgagaattgcttgaacctgggaggcagaggttgcagtgagctgagatcgccccactgcaatccagcctgggagacataattcaaatctattttggtcttatatct

SEQ ID NO: 36 - DNA, 253 bp, Homo sapiens
ctctctttttctaatgtaaatgttgtgtacaatagttttatttgattaagcttcaggactgttttgtaaagcgaggtgggaccgatgtggcacacgccagctgcggtttcccggagcgtggagaggcagtgctgctgctcccgcccgaggctcatgacaactcaataaagcactgcttttattttttgcagtcttcaatttgagaaaggtgagaaataatgttttccaataaatgagattcataccattaaaa

SEQ ID NO: 37 - DNA, 1884 bp, Homo sapiens
tctggtggcccttccggccacccctttaaccccagctttccctcccccttctttcgatcagagatcggcggagaccctcgaagtgcgcaaacttgacactcaccctgaccggactggggttttaaggggtgtggcaggaggttttggactcgatgagtttccaccgaaatgtcggagaagtcaggccagagcacaaaagcaaaggatgggaaaaagtatgcaacactcagtttatttaatacttacaaggggaaatcattagaaacacagaaaaccacagctcgacatggattacagagtcttggaaaagtcggtatttcacggcgtatgcctccacctgctaacctcccaagtcttaaagcagaaaacaaaggcaatgatcctaatgtaaacattgtacctaaagatggcacagggtgggcatcaaaacaagagcaacatgaagaagaaaaaacaccagaagtgccaccagcacagccaaaacctggggttgcagctcccccagaagtagcacctgctcccaaatcatgggccagtaacaagcaaggtgggcaaggagatggaatccaagtgaatagtcagtttcagcaagaatttcccagcctgcaggcagctggggatcaggaaaaaaaagaaaaggaaacaaatgatgacaactatggacctggacccagtttacgtccaccaaatgttgcttgttggagagatggtggtaaggctgctggctcaccttcgtcatctgatcaagatgaaaagctccctggccaggatgaaagcacagctggaacatcagagcaaaatgatatcctcaaagtggtggaaaagaggatagcttgtggtcctccacaggctaaactgaatggacagcagactgctctcgcttcccagtatagagctatgatgcctccttatatgttccaacagtatccgaggatgacatatcctcctctacatggtcccatgagattcccaccttctttatctgaaacaaacaaaggccttcgaggaagaggcccacctccttcatgggcctctgagcctgaacgcccatccattcttagtgcatcagaactgaaggagcttgataaatttgataacctagatgctgaagctgatgaaggttgggcaggtgctcagatggaagtagattatacagagcaactgaatttcagtgatgatgatgaacaaggaagtaacagtcctaaagagaataacagtgaggatcaaggttcaaaagcctctgaaaacaacgaaaacaaaaaagaaacagatgaagtttccaacactaaatcatcttcccaaatacctgcccaaccatcagtagcaaaagttccctatgggaaaggaccttcatttaatcaggaacgtggaacatcttcacatctgccaccacctccaaagttgcttgcacagcagcatccacctccagatcgacaggcagtacctggaagaccaggcccctttccctccaagcagcaagtagctgatgaagatgaaatatggaagcaaagacgaagacaacaatcagaaatttctgcagcagtagaacgtgctcgtaaacggcgtgaagaggaagagcgaagaatggaagaacaaaggaaggcagcttgtgcggagaaactgaaacgattggatgagaagcttggcatcctggaaaaacaaccatctccagaggaaattagggaaagggagcgagaaaaagaacgggagcgtgagaaagaacttgaaaaagaacaagaacaggagcgagagaaggagagggaaaaagacagagagagacagcaggaaaaggagaaagagctggagaaggagcaggaaaaacaaagagaaatggagaaagaaagaaagcaagaaaaagaaaaagaactagaacggcagaaaaaa

SEQ ID NO: 38 - DNA, 1004 bp, Homo sapiens
cgcaaaattaaccccctaataaaattaattaaccactcattcatcgacctccccaccccatccaacatctccgcatgatgaaacttcggctcactccttggcgcctgcctgatcctccaaatcaccacaggactattcctagccatgcactactcaccagacgcctcaaccgccttttcatcaatcgcccacatcactcgagacgtaaattatggctgaatcatccgctaccttcacgccaatggcgcctcaatattctttatctgcctcttcctacacatcgggcgaggcctatattacggatcatttctctactcagaaacctgaaacatcggcattatcctcctgcttgcaactatagcaacagccttcataggctatgtcctcccgtgaggccaaatatcattctgaggggccacagtaattacaaacttactatccgccatcccatacattgggacagacctagttcaatgaatctgaagaggctactcagtagacagtcccaccctcacacgattctttacctttcacttcatcttgcccttcattattgcagccctagcagcactccacctcctattcttgcacgaaacgggatcaaacaaccccctaggaatcacctcccattccgataaaatcaccttccacccttactacacaatcaaagacgccctcggcttacttctcttccttctctccttaatgacattaacactattctcaccagacctcctaggcgacccagacaattataccctagccaaccccttaaacacccctccccacatcaagcccgaatgatatttcctattcgcctacacaattctccgatccgtccctaacaaactaggaggcgtccttgccctattactatccatcctcatcctagcaataatccccatcctccatatatccaaacaacaaagcataatatttcgcccactaagccaatcactttattgactcctagccgcagacctcctcattctaacctgaatcgg

SEQ ID NO: 39 - DNA, 1129 bp, Homo sapiens
aaagtgtgagagggtccgtagttgggtcaactttgactcctctcgcctgcccggatccttaagggcctcctcgtcctcccggtctccggtcgctgccgggtctgtgcgccggtccgcgcccgccctcgctctgccatgggcgcttccagctcctccgcgctggcccgcctcggcctcccagcccggccctggcccaggtggctcggggtcgccgcgctaggactggccgccgtggccctggggactgtcgcctggcgccgcgcatggcccaggcggcgccggcggctgcagcaggtgggcaccgtggcgaagctctggatctacccggtgaaatcctgcaaaggggtgccggtgagcgaggctgagtgcacggccatggggctgcgcagcggcaacctgcgggacaggttttggctggtgattaaggaagatggacacatggtcactgcccgacaggagcctcgcctcgtgctcatctccatcatttatgagaataactgcctgatcttcagggctccagacatggaccagctggttttgcctagcaagcagccttcctcaaacaaactccacaactgcaggatatttggccttgacattaaaggcagagactgtggcaatgaggcagctaagtggttcaccaacttcttgaaaactgaagcgtatagattggttcaatttgagacaaacatgaagggaagaacatcaagaaaacttctccccactcttgatcagaatttccaggtggcctacccagactactgcccgctcctgatcatgacagatgcctccctggtagatttgaataccaggatggagaagaaaatgaaaatggagaatttcaggccaaatattgtggtgaccggctgtgatgcttttgaggaggcttcagcaaccaggagggattgactgagatcttaacaacagcagcaacgatacatcagcaaatccttattatccagccttcaactatctttaccctggaaaacaatctcgatttttgacttttcaaagttgtgtatgctccaggttaatgcaaggaaagtattagaggggggaatatgaaagtatatatataaattttaggtactgaaggctttaaaaataattaagatcatcaaaaatgcta

SEQ ID NO: 40 - DNA, 821 bp, Homo sapiens
gtcaactatgctggctggactggctgccttgttcctggcctaggacttagcttcataactatcacctgcaccgactaggctgaggtgctggtacttgccccaacccctacttttgtatttatatgtgtgtgtgtgtgtgcgtgcgtgcgtgcgtgcgtgtatgtttggtctggaccagcttctgccagcccctggcctttactttcttccttgcctatgcagggcaaacaaaatgtgaaattctgccctcagctgagctgagtaagggctcctgggggttggctggagatgggtgtggcatctgtccaggcctggaaccgtctcaagacagtgctggcaaagctgcagtattgagatgctaaggagctgatgccacctctttgtcttcccctaaaggagaacatggggataacatgggtgtgtgcccacaacactctaggtgcagagcccctgtggcaaagtattacagggtgtgggtggggattaccctgaatcggggattttaatgatggaagcaggcagagcctggtgggtgattctgtcaacagaaaattgcaatcatgcaggggctgggagggttaggatgaaaaaactggggccattggaggcccactgtaggtgggagggagctgattttggggtggggggtgggactagagggcaatactgaaggggttaaacaggtttttgctcctcaagaatttgtttgcctgggcccaggattggagggcttcacaccaataccctgtgtatacaagaatcagatttataatacttccccttttttgttacgtatgaacactataaaccaaattattttg

SEQ ID NO: 41 - DNA, 544 bp, Homo sapiens
tcatttggctcactatgaaaagcctttaattaatctcttcaaacaagttatttccttaatccacaagcagtggttacacttgctttgcattcttgtctggttcctaactctagagcccttctccctggcttagccagtaagctgagcccctggctgcgttcagccggcccgcctgagagacactaggggaaatagcttttgtgggcaagcagggtggccggtggtgctcagcagtctttccagtggctgtgtccctcctccaaatgtggacaggccatgacagagtcttagcccaagtcccacagatccccaaaagttctgttgattgcttcaggggatcagtgaaaattagggaattttgtgtgttgctatatacattttttctggggagatgagcttctcattgagatctgtgactcagaatcgactaagccaccataagtctggatttctccccagctcccaaggcccttttgggtccagaagacctgcatatgggctgttgactcatgcaaatgaggtatctgaactgcagcttcagtat

SEQ ID NO: 42 - DNA, 1067 bp, Homo sapiens
cctgtcagtcgccatcatacctcgagtgaggcccagctagataatgacttgtccaagatggcacacgtggaaagttgatctgcaccagaacccggatgactgtcaccttgaagcgtcctgttctccttctgtgctgtcccaggaagtgtctggcgggcgtgggcagcacagctctacactgtacgattcactagggcatcctgcgagcctcactagccttctggttcatgcctttgacaagcatttttgtgccccctctgcttactgtgacagtcgatgatgaatcttgcgttgccattttctgctgtgggtaactgcgtgcagtgtcttgccttgctttctcttcttactgtcccacagcttggtttcatgttacaaacagaaaagctcgaggctcccaccccgccacatcccaacttcatttccccctcactgtagcccatttccaccccaccacaaagttgccacaggttttctttgtatagaatatttattttgaagctctattttaatagtatttattttagaaagtctactattgtaagagttcttctgtttgtgaagaaaaaaacaagttaaaaactgaatgtactgatttagaaaatatatataaatatatattgttaaatatactttgattgcgccactgcactccagccttggcgaccagactaagacgctgtctcaaaaaaaaacaaaaacgacaaaaaaaaaacaaaacagaaaaaataaactaaggcaatgacagtccctggcaaatgctgggagggaggcagcagtggtcagggaaggtaaccctgaagcaggacttgtaaagcaaataagattgggaggccaaggtgggtggatcacgaggtcaggagttcgagaccagcctggccaacatagtgaaaccccgtctttactaaaaatacaaaaaaattagccaggtgtggtggtgggtgcctgtagtcccagctacttgggaggctgaggcaggagaatctcgaacccaggaggcggaggttacagtcagctgagaccgcaccattgcactccagcctgggtgacagagcaagattccgtct

SEQ ID NO: 43 - DNA, 1560 bp, Homo sapiens
ttaagggaaaaaatgactggctatattcagtcagatatggcaaaaagtctcaaggtgttaatgtgaatgattaaggtcttggggggggtgtcccctatcagactacaggtgtttagaggcacagaaaaaggtgcagttgggttcttaatgtgaaatgatgagaagcacaactccagtgtgtctctttgtgtagaatgtcagcagacaccccctgctagatgtgctggatcatgggaaagcatttccatttgttactagattgttcagaagttttaatttatgatgggtgtggtggctcatgcctgtagtcccagcactgtgggaggctgaggcaggaggatcatctgaggccaagagttcaagatcagcctgggcaacatagtgataccctatctcttaaaaaagaagaagtttttaaatttgaaataataataggtactggatttatgcaaatgtcttttctgcgtcttttgagatgagtatcaggtttttttttttccttttatcatctgatgatgaacttaatgtttccatttgtattaatggaatactaagtccctctgtgatttctgaaccaagctattcctaggcctgagttttattttgttgacacagaaataaattagaaggccaagcgtggtggcatgtgcctgtagtcctagttgctgaggtaagaggattgcttgagcccaggagttcaaggctgcagcaagctttgattgcgccactgcactccagccttggcgacagactaagacgctgtctcaaaaaaaaacaaaaacgacaaaaaaaaaacaaaacagaaaaaataaactaaggcaatgacagtccctggcaaatgctgggagggaggcagcagtggtcagggaaggtaaccctgaagcaggacttgtaaagcaaataagattgggaggccaaggtgggtggatcacgaggtcaggagttcgagaccagcctggccaacatagtgaaaccccgtctttactaaaaatacaaaaaaattagccaggtgtggtggtgggtgcctgtagtcccagctacttgggaggctgaggcaggagaatctcgaacccaggaggcggaggttacagtcagctgagaccgcaccattgcactccagcctgggtgacagagcaagattccgtctcaaaaaaaaaaaaaaaaaaaaaaccaagaagaaaaggaatgaattagaacttcttctgcttggacttaagggcatcatcaggcaggttttgggtaggatagcaggggaggcagagacatagtcggggtcagtggtcatgagtgtggctttgagcccaaaaacttggtttctgttccctactttgccactcagtagtgcatgactttggccaaatttcttaaattcatgaagcaagtttccgggtgaatgaaatggggataaaaatagtgttcaaacctatccgttggtttgtgtgaaactgaaatgaatagtatcgtgcaggtacttgtgagcaaggggagctgctgtttcctgtccctttatgatgggaaatatctagacaagttcccaaccctctgcactgcaggctgcat

SEQ ID NO: 44 - DNA, 397 bp, Homo sapiens
agagtcactatccggaaatctaagaatatcctctttgtcatcacaaaaccagatgtctacaagagccctgcttcagatacttacatagtttttggggaagccaagatcgaagatttatcccagcaagcacaactagcagctgctgagaaattcaaagttcaaggtgaagctgtctcaaacattcaagaaaacacacagactccaactgtacaagaggagagtgaagaggaagaggtcgatgaaacaggtgtagaagttaaggacatagaattggtcatgtcacaagcaaatgtgtcgagagcaaaggcagtccgagccctgaagaacaacagtaatgatattgtaaatgcgattatggaattaacaatgtaaccatatggaagcaactttttttggt

SEQ ID NO: 45 - DNA, 931 bp, Homo sapiens
gcctagccatgtgatttcacttccactccataacgctcctcatactaggcctactaaccaacacactaaccatataccaatgatggcgcgatgtaacacgagaaagcacataccaaggccaccacacaccacctgtccaaaaaggccttcgatacgggataatcctatttattacctcagaagtttttttcttcgcaggatttttctgagccttttaccactccagcctagcccctaccccccaattaggagggcactggcccccaacaggcatcaccccgctaaatcccctagaagtcccactcctaaacacatccgtattactcgcatcaggagtatcaatcacctgagctcaccatagtctaatagaaaacaaccgaaaccaaataattcaagcactgcttattacaattttactgggtctctattttaccctcctacaagcctcagagtacttcgagtctcccttcaccatttccgacggcatctacggctcaacattttttgtagccacaggcttccacggacttcacgtcattattggctcaactttcctcactatctgcttcatccgccaactaatatttcactttacatccaaacatcactttggcttcgaagccgccgcctgatactggcattttgtagatgtggtttgactatttctgtatgtctccatctattgatgagggtcttactcttttagtataaatagtaccgttaacttccaattaactagttttgacaacattcaaaaaagagtaataaacttcgccttaattttaataatcaacaccctcctagccttactactaataattattacattttgactaccacaactcaacggctacatagaaaaatccaccccttacgagtgcggcttcgaccctatatcccccgcccgcgtccctttctccataaaattctt

SEQ ID NO: 46 - DNA, 968 bp, Homo sapiens
gagcacatggttattactgatcgcattgaaaacattgatcacctgggtttctttatttatcgactgtgtcatgacaaggaaacttacaaactgcaacgcagagaaactattaaaggtattcagaaacgtgaagccagcaattgtttcgcaattcggcattttgaaaacaaatttgccgtggaaactttaatttgttcttgaacagtcaagaaaaacattattgaggaaaattaatatcacagcataaccccaccctttacattttgtgcagtgattattttttaaagtcttctttcatgtaagtagcaaacagggctttactatcttttcatctcattaattcaattaaaaccattaccttaaaatttttttctttcgaagtgtggtgtcttttatatttgaattagtaactgtatgaagtcatagataatagtacatgtcaccttaggtagtaggaagaattacaatttctttaaatcatttatctggatttttatgttttattagcattttcaagaagacggattatctagagaataatcatatatatgcatacgtaaaaatggaccacagtgacttatttgtagttgttagttgccctgctacctagtttgttagtgcatttgagcacacattttaattttcctctaattaaaatgtgcagtattttcagtgtcaaatatatttaactatttagagaatgatttccacctttatgttttaatatcctaggcatctgctgtaataatattttagaaaatgtttggaatttaagaaataacttgtgttactaatttgtataacccatatctgtgcaatggaatataaatatcacaaagttgtttaactagactgcgtgttgtttttcccgtataataaaaccaaagaatagtttggttcttcaaatcttaagagaatccacataaaagaagaaactattttttaaaaattcacttctatatatacaa

SEQ ID NO: 47 - DNA, 904 bp, Homo sapiens
ctgggggagaagagcctgccgaggaggactccgaggactggtgcgtgccctgcagcgacgaggaggtggagctgcctgcggatgggcagccctggatgcccccgccctccgaaatccagcggctctatgaactgctggctgcccacggtactctggagctgcaagccgagatcctgccccgccggcctcccacgccggaggcccagagcgaagaggagagatccgatgaggagccggaggccaaagaagaggaagaggaaaaaccacacatgcccacggaatttgattttgatgatgagccagtgacaccaaaggactccctgattgaccggagacgcaccccaggaagctcagcccggagccagaaacgggaggcccgcctggacaaggtgctgtcggacatgaagagacacaagaagctggaggagcagatccttcgtaccgggagggacctcttcagcctggactcggaggaccccagccccgccagccccccactccgatcctccgggagtagtctcttccctcggcagcggaaatactgattcccactgctcctgcctctagggtgcagtgtccgtacctgctggagcctgggccctccttccccagcccagacattgagaaacttgggaagaagagagaaacctcaagctcccaaacagcacgttgcgggaaagaggaagagagagtgtgagtgtgtgtgtgtgttttttctattgaacacctgtagagtgtgtgtgtgtgttttctattgaacacctatagagagagtgtgtgtgttttctattgaacatctatatagagagagtgtgtgagtgtgtgttttctattgaacacctattcagagacctggactgaattttctgagtctgaaataaaagatgcagagctatcatctctt

SEQ ID NO: 48 - DNA, 877 bp, Homo sapiens
aaaggacagtctcttcaataaatggtgttgggaaaactggatatccatatgcaaaagaatgaaattggacctttatctcacaccattagcaaaaatcagctcaaaatgaattaaagacttaaacataagacctgaaattgtaaaactactggaagaaaacgggaaaagttctaccacatttgtctgcaaaatgtcatatatattcaacagaatactattcagccctagaaaagaaggaaatcctgtcatttgtgacaatatgaatgaacctggaagacattatattaagcaaaataagccaggcatggaaagacaatattgcatgatctcacttatatgtggaatctaaaaaaaattggacttacaaaagaaaaaaagagaatggtggttactagatgccggggatgggaatgcggatggggaatgcagagatgttgatcaaaaggtacaaagtttcagctgggtgcagtggctcacgcctgtaatcccagcactttgggaggccaaggcaggcagatcacttgaggtcaggagtttgagaccagcctggccaacatggtgaaaccctgtccttgctaaaataaaaaaattagcccagtgtggtggcatgcacctgtaatcccagctactcaggaggtcgaggcggcagaattgcttgaacccaggaggtggaggttgtagtgagctgagattgtgccactgcactccagcctgggtgacagagcaagactctgtctcaaaaaaaaaaaagtacgaagtttcagttagacaggaaaagtgagttttcaggacctattgaacagcatggtgaccatagttaataataatatatatttcagattgctaaaagagcttattttaagtgttcttactacaa

SEQ ID NO: 49 - DNA, 793 bp, Homo sapiens
acgttgtaggcccctacgggctactacaacccttcgctgacgccataaaactcttcaccaaagagcccctaaaacccgccacatctaccatcaccctctacatcaccgccccgaccttagctctcaccatcgctcttctactatgaacccccctccccatacccaaccccctggtcaacctcaacctaggcctcctatttattctagccacctctagcctagccgtttactcaatcctctgatcagggtgagcatcaaactcaaactacgccctgatcggcgcactgcgagcagtagcccaaacaatctcatatgaagtcaccctagccatcattctactatcaacattactaataagtggctcctttaacctctccacccttatcacaacacaagaacacctctgattactcctgccatcatgacccttggccataatatgatttatctccacactagcagagaccaaccgaacccccttcgaccttgccgaaggggagtccgaactagtctcaggcttcaacatcgaatacgccgcaggccccttcgccctattcttcatagccgaatacacaaacattattataataaacaccctcaccactacaatcttcctaggaacaacatatgacgcactctcccctgaactctacacaacatattttgtcaccaagaccctacttctaacctccctgttcttatgaattcgaacagcatacccccgattccgctacgaccaactcatacacctcctatgaaaaaacttcctaccactcaccctagcattacttat

SEQ ID NO: 50 - DNA, 2086 bp, Homo sapiens
tattttcagtagagacggggtttcaccatgttagccaggatggtctcgatctcctgacctcgtgatccgcccgcctcggcctcccaaagtgctgggattacaggcatgagccaccgggcccggatgtcttggtttttcttgatatttgaaacagtaggtcaaaattggacatgtctagtaggcggttgctggtttagtaataaagggtagtagctgtaattgcccagtttgaatagtgttcatgtttttaaggatttcacaaacaaatcttcatcgaataaatgtttttaaaaaatgattactgattagattcaatataaattaaggaaggtcccatcatatctcttgcacaaatcattgccttccagaattaagcacagcatcgatatgtatgtcttacatgacatattgctctgcaatgtgatggaatcaacagaacaagatgtttggagtcagatggctaaactaggatttaaagttcacattatttactagttaactgtggatgctaaggcaatattcttgaagtctccatgtcttgatttcctcttagtgaaatgggaatcataataataataattttacagggttggtgtaaaaacgaaatgactgcctgcactcaatacaatacctattatggttatgttagttatattatgattaatgtattagtatactattgataaaatgaatatgaaattgctagtagccagctcacaatattagtatgacaaaaattatttcaatttggtttaaatacaatacctgtggtcaataacatcatcttttcttaaaacttaatgcactgagcactgtcaattacaatgaaaaaaaaatgcaagtaacccagttggactatccattaactgagaaaatataatatgaaaaagccaccatgaattcaggaccacttatgaatgtagctgtattttattgatagaatttatatgtgcttagaaaaaacatagtacattttatgttagatatatttattcagattaccaaagaaaatgcttactgtacatatggattgatgggccctatccaataactttcatcactcctcaagggcccttggcacacaggctgggaatcactgatgttggttaaggtcacaatagacaggggagcctcatgcttataattctgcttgccaagtggaaatgctaacctgctctttcccaggattttagaaaaacatttgaaatatttcacaagataaatgaaaatgggtaaacacaaggccttaaggaaaatatacttctggtgcatttattaagcacttggatctgatgcatgacacctgcttaaaactcaaagaattatcacaggctttgctgaggaagaaagaacaagaattatgactgcacacaggctcatacttcagcaagtcaatgtttttaaatcaatgaaaaacaatccagtgattgtgtttgctacaaagggaaagcaatgtaagatgacatacagtatttcaagctgttcaaattactgatggcacagtggcaagatatatatccattaaaaccaaatagttgtttcaaagtatgttggccaacagagataaataatgtttctctgaagtgaatataaaataatgtctctcttcagaatgtaggaccctataatggctttagccacaaatacctcttgcttatttggggaatacgcattttccaccttcattcagaaattctttagttagctcaacaaaattcatctttcacctcaacaaaaaagcatgcgcattaagtatcaaagtatgtatatttctacagtaggctcactgatggtctcattcctattggtaaatttgtcatagtgaatgagtaaagtatgaataaatacaatgtgttgaatgtagaggctactttcacaagaatattgcatcatcagtgggccaatgttaagagcatgtttgtgaggaagaggaacatacaatttctctttttttcttaattcttctcttatcagcattttaattaataaacataaaaaggtaacactgatacacaaagtattaaatcaccatccattcgtctttaaagatttgcattctcctaatcagtgaaaacaattctttctt

SEQ ID NO: 51 - DNA, 941 bp, Homo sapiens
aaataaacatgctagcttttattccagttctaaccaaaaaaataaaccctcgttccacagaagctgccatcaagtatttcctcacgcaagcaaccgcatccataatccttctaatagctatcctcttcaacaatatactctccggacaatgaaccataaccaatactaccaatcaatactcatcattaataatcataatggctatagcaataaaactaggaatagccccctttcacttctgagtcccagaggttacccaaggcacccctctgacatccggcctgcttcttctcacatgacaaaaactagcccccatctcaatcatataccaaatctctccctcactaaacgtaagccttctcctcactctctcaatcttatccatcatagcaggcagttgaggtggattaaaccaaacccagctacgcaaaatcttagcatactcctcaattacccatataggatgaataatagcagttctaccgtacaaccctaacataaccattcttaatttaactatttatattatcctaactactaccgcattcctactactcaacttaaactccagcaccacgaccctactactatctcgcacctgaaacaagctaacatgactaacacccttaattccatccaccctcctctccctaggaggcctgcccccgctaaccggctttttgcccaaatgggccattatcgaagaattcacaaaaaacaatagcctcatcatccccaccatcatagccaccatcaccctccttaacctctacttctacctacgcctaatctactccacctcaatcacactactccccatatctaacaacgtaaaaataaaatgacagtttgaacatacaaaacccaccccattcctccccacactcatcgcccttaccacgctactcctacctatctccccttttatactaataatctta

SEQ ID NO: 52 - DNA, 517 bp, Homo sapiens
ttaaaaatgccctagcccacttcttaccacaaggcacacctacaccccttatccccatactagttattatcgaaaccatcagcctactcattcaaccaatagccctggccgtacgcctaaccgctaacattactgcaggccacctactcatgcacctaattggaagcgccaccctagcaatatcaaccattaaccttccctctacacttatcatcttcacaattctaattctactgactatcctagaaatcgctgtcgccttaatccaagcctacgttttcacacttctagtaagcctctacctgcacgacaacacataatgacccaccaatcacatgcctatcatatagtaaaacccagcccatgacccctaacaggggccctctcagccctcctaatgacctccggcctagccatgtgatttcacttccactccataacgctcctcatactaggcctactaaccaacacactaaccatataccaatgatggcgcgatgtaacacgagaaagcaca

SEQ ID NO: 53 - DNA, 793 bp, Homo sapiens
ggcgaggcctatattacggatcatttctctactcagaaacctgaaacatcggcattatcctcctgcttgcaactatagcaacagccttcataggctatgtcctcccgtgaggccaaatatcattctgaggggccacagtaattacaaacttactatccgccatcccatacattgggacagacctagttcaatgaatctgaggaggctactcagtagacagtcccaccctcacacgattctttacctttcacttcatcttgcccttcattattgcagccctagcagcactccacctcctattcttgcacgaaacgggatcaaacaaccccctaggaatcacctcccattccgataaaatcaccttccacccttactacacaatcaaagacgccctcggcttacttctcttccttctctccttaatgacattaacactattctcaccagacctcctaggcgacccagacaattataccctagccaaccccttaaacacccctccccacatcaagcccgaatgatatttcctattcgcctacacaattctccgatccgtccctaacaaactaggaggcgtccttgccctattactatccatcctcatcctagcaataatccccatcctccatatatccaaacaacaaagcataatatttcgcccactaagccaatcactttattgactcctagccgcagacctcctcattctaacctgaatcggaggacaaccagtaagctacccttttaccatcattggacaagtagcatccgtactatacttcacaacaatcct

SEQ ID NO: 54 - DNA, 827 bp, Homo sapiens
ggaaccttctggttcctgcgccaatcgccaccagtatcaattgtgtgagcttgggtgcgagtgcacgcgtgcgtgagtacggagagtatatatagatctctatctcttagcaaaggtgaatgccagatgtaaatggcgcctctgggcaaaggaggcttgtattttgcacattttataaaaacttgagagaatgagatttctgcttgtatatttctaaaaagaggaaggagcccaaaccatcctctccttaccactcccatccctgtgagccctaccttacccctctgcccctagccaaggagtgtgaatttatagatctaactttcataggcaaaacaaaagcttcgagctgttgcgtgtgtgagtctgttgtgtggatgtgcgtgtgtggtccccagccccagactggattggaaaagtgcatggtgggggcctcggggctgtccccacgctgtccctttgccacaagtctgtggggcaagaggctgcaatattccgtcctgggtgtctgggctgctaacctggcctgctcaggcttcccaccctgtgcggggcacacccccaggaagggaccctggacacggctcccacgtccaggcttaaggtggatgcacttcccgcacctccagtcttctgtgtagcagctttaacccacgtttgtctgtcacgtccagtcccgagacggctgagtgaccccaagaaaggcttccccgacacccagacagaggctgcagggctggggctgggtgagggtggcgggcctgcggggacattctactgtgctaaaaagccactgcagacatagcaataaaaacatgtcattttcc

SEQ ID NO: 55 - DNA, 1562 bp, Homo sapiens
cccaccgactccactatgttgaagaaattcgacaagaaggatgaggagtcaggtggaggctccaacccattccagcaccttgagaagagtgcggtactccaggaggcccgtgtatttaatgaaactcccatcaaccctcggaaatgtgcccacatcctcaccaagattctttatctcataaaccagggggagcacctggggaccacggaagcgaccgaggccttctttgccatgaccaagctctttcagtccaatgatcccacactccgtcggatgtgctacttgaccatcaaggagatgtcttgcattgcagaggatgtcatcattgtcaccagcagcctaacaaaagacatgactgggaaagaagacaactaccggggcccggccgtgcgagccctctgccagatcactgatagcaccatgctgcaggctattgagcgctacatgaaacaagccattgtggacaaggtgcccagtgtctccagctctgccctcgtgtcttccttgcacctgctgaagtgcagctttgacgtggtcaagcgctgggtgaatgaggctcaggaggcagcatccagtgataacatcatggtccagtaccacgcactagggctcctgtaccatgtgcgtaagaatgaccgcctagccgtcaataagatgatcagcaaggtcacacggcatggccttaagtctccctttgcctactgcatgatgatccgggtggccagcaagcagctggaagaggaggatggcagccgtgacagcccactgtttgacttcatcgagagctgcttgcgcaacaagcacgagatggtggtgtatgaagccgcctcggccatcgtcaatctgccaggctgcagtgccaaagagctggccccggctgtgtcagtgctccagcttttctgcagctcacccaaggctgctctccgctatgctgctgttcgtaccctcaataaggttgccatgaagcatccgtcagctgtgacagcttgtaatctggatctggagaacctggtcacagattcaaaccgcagcattgccacgctggccatcaccaccctccttaagacgggcagcgagagcagcatcgaccgcctcatgaagcagatctcctccttcatgtcagaaatctcggatgaattcaaggtggtggttgtccaggccatcagtgccctgtgtcagaaatatcctcgcaaacacgccgtccttatgaacttcctgttcaccatgctgcgggaagagggtggctttgagtataagcgcgctatcgtggactgcatcatcagcatcattgaagagaactcagagagcaaggagacagggctgtcacatctgtgcgagttcatcgaggactgcgagttcacagtgctggccacccgtattctacatctcctgggccaggaggggcccaagaccaccaatccctcaaagtacatccgcttcatctataaccgagtggtcttggagcatgaggaggtccgggcaggtgctgtgagtgctctggcgaagtttggagcccagaatgaagagatgttacccagtatcttggtgttgctgaagaggtg

SEQ ID NO: 56 - DNA, 678 bp, Homo sapiens
ccccaaacccactccaccttactaccagacaaccttagccaaaccatttacccaaataaagtataggcgatagaaattgaaacctggcgcaatagatatagtaccgcaagggaaagatgaaaaattataaccaagcataatatagcaaggactaacccctataccttctgcataatgaattaactagaaataactttgcaaggagagccaaagctaagacccccgaaaccagacgagctacctaagaacagctaaaagagcacacccgtctatgtagcaaaatagtgggaagatttataggtagaggcgacaaacctaccgagcctggtgatagctggttgtccaagatagaatcttagttcaactttaaatttgcccacagaaccctctaaatccccttgtaaatttaactgttagtccaaagaggaacagctctttggacactaggaaaaaaccttgtagagagagtaaaaaatttaacacccatagtaggcctaaaagcagccaccaattaagaaagcgttcaagctcaacacccactacctaaaaaatcccaaacatataactgaactcctcacacccaattggaccaatctatcaccctatagaagaactaatgttagtataagtaacatgaaaacattctcctccgcataagcctgcgtcagattaaaacac

SEQ ID NO: 57 - DNA, 1338 bp, Homo sapiens
tcacaacaagcaaacctggagtatccttggtctactccatgccctcccggaacctgtccctgcggctggagggtctccaggagaaagactctggcccctacagctgctccgtgaatgtgcaagacaaacaaggcaaatctaggggccacagcatcaaaaccttagaactcaatgtactggttcctccagctcctccatcctgccgtctccagggtgtgccccatgtgggggcaaacgtgaccctgagctgccagtctccaaggagtaagcccgctgtccaataccagtgggatcggcagcttccatccttccagactttctttgcaccagcattagatgtcatccgtgggtctttaagcctcaccaacctttcgtcttccatggctggagtctatgtctgcaaggcccacaatgaggtgggcactgcccaatgtaatgtgacgctggaagtgagcacagggcctggagctgcagtggttgctggagctgttgtgggtaccctggttggactggggttgctggctgggctggtcctcttgtaccaccgccggggcaaggccctggaggagccagccaatgatatcaaggaggatgccattgctccccgaaccctgccctggcccaagagctcagacacaatctccaagaatgggaccctttcctctgtcacctccgcacgagccctccggccaccccatggccctcccaggcctggtgcattgacccccacgcccagtctctccagccaggccctgccctcaccaagactgcccacgacagatggggcccaccctcaaccaatatcccccatccctggtggggtttcttcctctggcttgagccgcatgggtgctgtgcctgtgatgatgcctgcccagagtcaagctggctctctggtatgatgaccccaccactcattggctaaaggatttggggtctctccttcctataagggtcacctctagcacagaggcctgagtcatgggaaagagtcacactcctgacccttagtactctgcccccacctctctttactgtgggaaaaccatctcagtaagacctaagtgtccaggagacagaaggagaagaggaagtggatctggaattgggaggagcctccacccacccctgactcctccttatgaagccagctgctgaaattagctactcaccaagagtgaggggcagagacttccagtcactgagtctcccaggcccccttgatctgtaccccacccctatctaacaccacccttggctcccactccagctccctgtattgatataacctgtcaggctggcttggttaggttttactggggcagaggatagggaatc

SEQ ID NO: 58 - DNA, 1999 bp, Homo sapiens
cccatttttgtatacctatcatccttccaataaatcactctttgcttagtttaaccagactcagtttctgttgctctcaactaaaacagttaacagataatgattctatattaaaatctcttagaatttcttttctatatttttattgggtatttatcatgttagcagagcctgtaaactttaagggcttcaagaaatttttaaaaaatatttcccggccagatgcggtggctcacacctgtaatactagcactttgggaggccaaggcgggcggatcacctgaggtcaggagtttgagaccagcctgaccaacatggagaaaccccatttctactaaaaatacaaaattagccgggcgtggtggtgcatgcctgtaatcccagctactcgggaggctgaggcaggagaattgcttgaacctgggaggcggaggttgcggtgagtcaagatcctgcaaccacattccggcctgggcaaaaaagcaagactctttctcaaaaaaaaaaaaaaaaaaaaggtaaaatgacaaagtcatgcacacacatgctaatctggaaagctataaatcaaactgaaaacactggttatcttgtagtggaatggctggccactttgactttctactttaaacatttggcctatttatttattttaaagagacagggtctcactctgttgctgcccaagctggagtagagtggcgtaaccatagctcactgtaacctcaaactcctgggctcatgtaatcctctcacttcagcctccagagtagctgagactatacaagtgcccgccaccatacctggttaatttttttttttttttagatgaggtttccctatgttgcccaggctggtcttgaactcctggcctcaaggaatcctcccagtttggccttccaaagtgctggaattgcaggcataagccaccatgcctggcccagaacaaacttattcttatgtcatgtttgtggcaacaaaaattcaaaaaataatgagacttccaggttagctttaatagagagaggcaagctagtgctttcacagtaacaactagataaaactaaatagattattcaaataaataaatggctttaaagattgaagagctgtcttctatgcatagcaaagaggactagcagaattaaaattccagagggagaagaggcctctctaggtgagctgaaagtgccagccatttcctctctctacttgtggtacttgtagagctttagcgtgagtgatgataactggggcttgccataggcagaactccatcaagagaaagaaaaaccagcagaacttatgacagctatatgggttggcatgacagactaaatttcataacagccctaaatttatggccagttttctacacaggacattcacctccaggctggaggcctgaactagaaaaccaaagtgaaattaagcacaatctcacagtgcttagcagataaagctagagggagtggcctgtaacaaacatacaacggatcccactcttgggatactacaccgaactgaaggacttaaatcttcagcccagtccaactcagttgtttggatagaggtgttttatacctcattgtatctgcctaagactgcaaaatgcaaacattaactttagactctaaaggtctttacacacattgtctagaaatcgtcaaaaacttctgctgcctggcgcagtggctcacgtctgtagttccagcattttgggaggccgaggcaggctgatcatgaggtcaggacttcgagaccagcctgaccaacatggtgaaaccctctctctactgaaaatacaaaaatttgccaggcatggtggcgcacgcccataatcccagctactggggaggctgaggcaggagagccgcttgaacccaggaggcaaaggttgcagtgagccaaaaccgcgtgccactgcactccagcctgggcaacagagcgagactctgtctcaaaaaacaacaacaacttttgctttcccaggagggag

SEQ ID NO: 59 - DNA, 957 bp, Homo sapiens
ggcctgctcaaggtggtgttcgtggtcttcgcctccttgtgtgcctggtattcggggtacctgctcgcagagctcattccagatgcacccctgtccagtgctgcctatagcatccgcagcatcggggagaggcctgtcctcaaaggtgagtgccgtgcttggggcagacggctgccttcatggcttgtgtgcagagggaggagtggtggtttctgtcccagctggaggctggggggccctgacggctttatatcaggaaggaggaggagagaagcattttgcagttactcatttaggtcccacgccattccaaggcacgtgccgctgctgaccccatgtgagactagaacagtgtggcttctggttaccagcacaaacctgcagccagaccttgaactgtgctctttccgcccagatcttggcttcctcccctgggaagcacaggagggtccaggttctgagatcccgaatcattttaaggtttctgtcggactgaagagctgttggaaaatcttctgtaaagtcttgggaagcaaaagctagtaattgagtagatgtgaaagaaagggcagacattgtatgtcggtgaacacacggcctcaaccatcagcttaggaaaggcccagctctctgtagggcggctgctttctgtccttccccacaatcaaggtgggttttctgagagcctgctgtgtgctgggcatgtccaggagctaaggagagacggaggacaagcccacgaggtccctgcccctttggcactgataccctagtgaggggaggtgacagcttaacgcatgaacaaatgagattcgagagaggatgggtggggaaagcctctgagaaggaggtgacaattgagaagcatctcagtgaagcaagggagtgggccatgctgggttctgagggctgtgtgccagtttgggagcatcatgacttagtctacccagattactg

SEQ ID NO: 60 - DNA, 941 bp, Homo sapiens
cttaggaaactggagaggatattcatggtcttaagcactagattatagagtttaggttgccagagtaacagtttcaaagatcctggcacacacagttgctctttagtaaatagttcttaaataaattgagacatgtacatttattctcaatttcctctttcttatttgttttccagagtgtttccttactctcagaagtttaaaaaattaagtacttggggctgggcgcagtgactcacgcctgtaatcccagcactttgggaggctgaggtggccagatcataaggtcaagagatcgagatcatcctggccaacatggtgaaaccccgtctctactaaaaatacaaaaactagctgagcgtggtggcaggcacctgtagtcccagctatttgggaggctgaggcagcagaatcacttgaacccaggaggggaggttgcagtgagccgagatcgcgccatgcacaccagcctgggtgatagagcgagactccatctccaaaaaaaaaaaaagtacttgggtacagtggctcacacctgtaatcctagcatttggggagaccaaggtgggaggatcacttgagcctgggaggtcaaggctgcagtgagacatgtttgagcactgcattccaacctgggtgacagagagcctatcttattgttgtataaaggtttgaaataggtacaaggtttggtcattcagtttcaaattcatttctcaaatatccttgtatctcactaacatcatctgtctaccagtaacatttccaaatctaatagtacttggcaagagatggggagggggttaaattgcacaatttaaaattttcataattgttatgcatcacagttattgtgatcacgtgataatgttatgcatgtcttttgttgtgtgtcagccccaacttttgtctcagcatttgaaccatgcatacatcttatc

SEQ ID NO: 61 - DNA, 2131 bp, Homo sapiens
agaagctgccatgacctgaattaggtaatgggtagtggacagaagggcacaactgcccatgcctgggagggaacaaaggggtgttgactcctgtgaacccacttcccaagctctatatcagagctcctgggctgcataggagtggccaggctaacaacctgaccccttcccttcctccctgggcagctccctgcggtgtggccccccaagcacgcatcacaggtggcagcagtgcagtcgccggtcagtggccctggcaggtcagcatcacctatgaaggcgtccatgtgtgtggtggctctctcgtgtctgagcagtgggtgctgtcagctgctcactgcttccccaggtaccaaatggtgaaagtcaaaagtgggctggaggtcaaagttaatgggtcaatccaggtcagaggttggggttcttgggttgggctgagagtgtcagttctaagtctggaaggaccagttaggagtgaaggtgaggggtcagaggttacatgataaaactaaggtgcaatttgagacctaaaaggggctgatcggaatcaggggttggcagtgggggttggggaacaaggagaggtctcctgggtctcagcctttgccccctgcctgcagcgagcaccacaaggaagcctatgaggtcaagctgggggcccaccagctagactcctactccgaggacgccaaggtcagcaccctgaaggacatcatcccccaccccagctacctccaggagggctcccagggcgacattgcactcctccaactcagcagacccatcaccttctcccgctacatccggcccatctgcctccctgcagccaacgcctccttccccaacggcctccactgcactgtcactggctggggtcatgtggccccctcaggtgaggtgggacgtgggtgcctagaggtgtggaggggcacctgacttggggaggggcccagggtaagcctcttttacccccacagtgagcctcctgacgcccaagccactgcagcaactcgaggtgcctctgatcagtcgtgagacgtgtaactgcctgtacaacatcgacgccaagcctgaggagccgcactttgtccaagaggacatggtgtgtgctggctatgtggaggggggcaaggacgcctgccaggtaagcacaggcccgggggcagatgaccagtgcaacttcggaaaggaggcctggcccggtcctgatggctgctgtgtggcttctctcctcagggtgactctgggggcccactctcctgccctgtggagggtctctggtacctgacgggcattgtgagctggggagatgcctgtggggcccgcaacaggcctggtgtgtacactctggcctccagctatgcctcctggatccaaagcaaggtgacagaactccagcctcgtgtggtgccccaaacccaggagtcccagcccgacagcaacctctgtggcagccacctggccttcagctctgccccagcccagggcttgctgaggcccatccttttcctgcctctgggcctggctctgggcctcctctccccatggctcagcgagcactgagctggccctacttccaggatggatgcatcacactcaaggacaggagcctggtccttccctgatggcctttggacccagggcctgacttgagccactccttccttcaggactctgcgggaggctggggccccatcttgatctttgagcccattcttctgggtgtgctttttgggaccatcactgagagtcaggagttttactgcctgtagcaatggccagagcctctggcccctcacccaccatggaccagcccattggccgagctcctggggagctcctgggacccttggctatgaaaatgagccctggctcccacctgtttctggaagactgctcccggcccgctgcccagactgatgagcacatctctctgccctctccctgtgttctgggctggggcacctttgtgcagcttcgaggacaggaaaggccccaatcttgcccactggccgctgagcgcccccgagccctgactcctggactccggaggactgagcccccaccggaactgggctggcgcttggatctggggtgggagtaacagggcagaaatgattaaaatgtttga

SEQ ID NO: 62 - DNA, 1032 bp, Homo sapiens
ctacgcggccgagatctgcatcgccctcaacttcctgcacgagagggggatcatctacagggacctgaagctggacaacgtcctcctggatgcggacgggcacatcaagctcacagactacggcatgtgcaaggaaggcctgggccctggtgacacaacgagcactttctgcggaaccccgaattacatcgcccccgaaatcctgcggggagaggagtacgggttcagcgtggactggtgggcgctgggagtcctcatgtttgagatgatggccgggcgctccccgttcgacatcatcaccgacaacccggacatgaacacagaggactaccttttccaagtgatcctggagaagcccatccggatcccccggttcctgtccgtcaaagcctcccatgttttaaaaggatttttaaataaggaccccaaagagaggctcggctgccggccacagactggattttctgacatcaagtcccacgcgttcttccgcagcatagactgggacttgctggagaagaagcaggcgctccctccattccagccacagatcacagacgactacggtctggacaactttgacacacagttcaccagcgagcccgtgcagctgaccccagacgatgaggatgccataaagaggatcgaccagtcagagttcgaaggctttgagtatatcaacccattattgctgtccaccgaggagtcggtgtgaggccgcgtgcgtctctgtcgtggacacgcgtgattgaccctttaactgtatccttaaccaccgcatatgcatgccaggctgggcacggctccgagggcggccagggacagacgcttgcgccgagaccgcagagggaagcgtcagcgggcgctgctgggagcagaacagtccctcacacctgggcccgggcaggccagcttcgtgctggaggaacttgctgctgtgcctgcgtcgcggcggatccgcggggaccctgccgagggggctgtcatgcggtttccaaggtgcacattttccacggaaacagaactcgatgc

SEQ ID NO: 63 - DNA, 615 bp, Homo sapiens
gtttctggatactgccttcaatctccaggcctttgagggcaagacattttagagctgggcaaaacctgtgtaggtctcgctgtgggtttgctggggaccaagggggtgatgaaaaggggaggggcggagctcctgcccaagagaggggctgtggggccccaggataaaacagacacagtgacagggccaagagccagcactgctggccttggtgtcatgccagaatctaccaggactgagggagccagaggagtcctgtaggcaggctactgtgctggagcatcccccagctgctcccatcttgctggaatttcttgggcggcttctccacctgtatctcaagacagacacccgggggcctgtgtctgtggccgctcccatcccggcagccctggctgctgctcgccccaccctcgcttatctgtagattcaaagcgatgttctcttctgtgctcttagaagtagggagttcagcagtaacagccaggtgaagcgaacctgctgggtgatttgtttgcgctctgttttatggggcattcctgcgagatgtgtcagcttctgtatgagatgcagccacagctcatgtgtaccaaagtagaaaaccaaatcacagag

SEQ ID NO: 64 - DNA, 123 bp, Artificial Sequence
  Description of Artificial Sequenceprimer
aggcggcaacgccgaggagaggagaggaaggccccagaaaaccaggaggaagaggaggagcgtgcagaactgaatcagtcggaggaacctgaggcaggcgagagtagtactggagggccttga

SEQ ID NO: 65 - DNA, 29 bp, Artificial Sequence
  Description of Artificial Sequenceprimer
acgtgaattcaggcggcaacgccgaggag

SEQ ID NO: 66 - DNA, 32 bp, Artificial Sequence
  Description of Artificial Sequenceprimer
cgatctcgagtcaaggccctccagtactactc

SEQ ID NO: 67 - protein, 72 aa, Homo sapiens
KCERVRSWVNFDSSRLPGSLRASSSSRSPVAAGSVRRSAPALALPWALPAPPRWPASASQPGPGPGGSGSPR

SEQ ID NO: 68 - protein, 13 aa, Homo sapiens
STMLAGLAALFLA

SEQ ID NO: 69 - protein, 25 aa, Homo sapiens
VNYAGWTGCLVPGLGLSFITITCTD

SEQ ID NO: 70 - protein, 35 aa, Homo sapiens
KDSLFNKWCWENWISICKRMKLDLYLTPLAKISSK

SEQ ID NO: 71 - protein, 11 aa, Homo sapiens
QPRPPIYSSHL

SEQ ID NO: 72 - protein, 49 aa, Homo sapiens
INMLAFIPVLTKKINPRSTEAAIKYFLTQATASIILLIAILFNNILSGQ

SEQ ID NO: 73 - protein, 25 aa, Homo sapiens
GEAYITDHFSTQKPETSALSSCLQL

SEQ ID NO: 74 - protein, 13 aa, Homo sapiens
RGLYYGSFLYSET

SEQ ID NO: 75 - protein, 37 aa, Homo sapiens
SQQANLEYPWSTPCPPGTCPCGWRVSRRKTLAPTAAP

SEQ ID NO: 76 - protein, 59 aa, Homo sapiens
TPTPSLSSQALPSPRLPTTDGAHPQPISPIPGGVSSSGLSRMGAVPVMMPAQSQAGSLV

SEQ ID NO: 77 - protein, 19 aa, Homo sapiens
KYKNLPGMVAHAHNPSYWG

SEQ ID NO: 78 - protein, 109 aa, Homo sapiens
ACSRWCSWSSPPCVPGIRGTCSQSSFQMHPCPVLPIASAASGRGLSSKVSAVLGADGCLHGLCAEGGVVVSVPAGGWGALTALYQEGGGEKHFAVTHLGPTPFQGTCRC

SEQ ID NO: 79 - protein, 35 aa, Homo sapiens
PAQGGVRGLRLLVCLVFGVPARRAHSRCTPVQCCL

SEQ ID NO: 80 - protein, 60 aa, Homo sapiens
KVKLLRSLPQRFKMDVHITPGTHASEHAVNKQLADKERVAAALENTHLLEVVNQCLSARS

SEQ ID NO: 81 - protein, 35 aa, Homo sapiens
QNIHLIRAPLAGKGKQLEEKMVQQLQEDVDMEDAP

SEQ ID NO: 82 - protein, 118 aa, Homo sapiens
FKPKPPKNESLETYPVMKYNPNVLPVQCTGKRDEDKDKVGNVEYFGLGNSPGFPLQYYPYYGKLLQPKYLQPLLAVQFTNLTMDTEIRIECKAYGENIGYSEKDRFQGRFDVKIEVKS

SEQ ID NO: 83 - protein, 98 aa, Homo sapiens
NESEVKGYKVLYRWNRQSSTSVIETNKTSVELSLPFDEDYIIEIKPFSDGGDGSSSEQIRIPKISNAYARGSGASTSNACTLSAISTIMISLTARSSL

SEQ ID NO: 84 - protein, 147 aa, Homo sapiens
GIQSTPLNLAVNWRCEPSSTDLRIDYKYNTDAMTTAVALNNVQFLVPIDGGVTKLQAVLPPAVWNAEQQRILWKIPDISQKSENGGVGSLLARFQLSEGPSKPSPLVVQFTSEGSTLSGCDIELVGAGYRFSLIKKRFAAGKYLADN

SEQ ID NO: 85 - protein, 76 aa, Homo sapiens
MSKAHPPELKKFMDKKLSLKLNGGRHVQGILRGFDPFMNLVIDECVEMATSGQQNNIGMVVIRGNSIIMLEALERV

SEQ ID NO: 86 - protein, 240 aa, Homo sapiens
PDPRPSPPRPDVCMADPEGLSSESGRVERLREKEKVQGRVGRRAPGKAKPASPARRLDLRGKRSPTPGKGPADRASRAPPRPRSTTSQVTPAEEKDGHSPMSKGLVNGLKAGPMALSSKGSSGAPVYVDLAYIPNHCSGKTADLDFFRRVRASYYVVSGNDPANGEPSRAVLDALLEGKAQWGENLQVKVTLIPTHDTEVTREWYQQTHEQQQQLNVLVLASSSTVVMQDESFPACKIEF

SEQ ID NO: 87 - DNA, 183 bp, Homo sapiens
aaggtcaagcttctgcgctcccttcctcagcgtttcaagatggacgtgcacattactccggggacccatgcctcagagcatgcagtgaacaagcaacttgcagataaggagcgggtggcagctgccctggagaacacccacctcttggaggttgtgaatcagtgcctgtcagcccgctcctga

SEQ ID NO: 88 - DNA, 108 bp, Homo sapiens
caaaacatccatcttatccgagcccctcttgcaggcaaagggaaacagttggaagagaaaatggtacagcagttacaagaggatgtggacatggaagatgctccttaa

SEQ ID NO: 89 - DNA, 357 bp, Homo sapiens
ttcaaacctaagcctcccaagaatgagtccttggagacttacccagtgatgaagtataacccaaatgtccttcccgttcagtgcactggcaagcgagatgaagataaggataaagttggaaatgtggagtattttggactgggcaactcccctggttttcctctgcagtattatccgtactatggcaaactcctgcagcccaaatacctgcagcccctgctggccgtacagttcaccaatcttaccatggacactgaaattcgcatagagtgtaaggcgtacggtgagaacattgggtacagtgagaaagaccgttttcagggacgttttgatgtaaaaattgaagttaagagctga

SEQ ID NO: 90 - DNA, 297 bp, Homo sapiens
aatgagtcggaagtaaaaggatacaaagtcttgtacagatggaacagacaaagcagcacatctgtcattgaaacaaataaaacatcggtggagctttctttgcctttcgatgaagattatataatagaaattaagccattcagcgacggaggagatggcagcagcagtgaacaaattcgaattccaaagatatcaaatgcctacgcgagaggatctggggcttccacttcgaatgcatgtacgctgtcagccatcagtacaataatgatttccctcacagctaggtccagtttatga

SEQ ID NO: 91 - DNA, 444 bp, Homo sapiens
ggcattcagtccacacctctgaacctggcagtgaattggcgatgtgagccttcaagcactgacctgcgcatagattacaaatataatacagatgcaatgacgactgctgtggccctcaacaatgtgcagttcctggtccccatcgacggaggagtcaccaagctccaggcagtgctcccaccagcagtctggaatgctgaacaacagagaatattgtggaagattcctgatatctctcagaagtcagaaaatggaggggtgggttctttgttggcaagatttcagttatctgaaggcccaagcaaaccttctccattggttgtgcagttcacaagtgaaggaagcaccctttctggctgtgacattgaacttgttggagcagggtatcgattttcactcatcaagaaaaggtttgctgcaggaaaatacttggcagataactaa

SEQ ID NO: 92 - DNA, 231 bp, Homo sapiens
atgagcaaagctcaccctcccgagttgaaaaaatttatggacaagaagttatcattgaaattaaatggtggcagacatgtccaaggaatattgcggggatttgatccctttatgaaccttgtgatagatgaatgtgtggagatggcgactagtggacaacagaacaatattggaatggtggtaatacgaggaaatagtatcatcatgttagaagccttggaacgagtataa

SEQ ID NO: 93 - DNA, 723 bp, Homo sapiens
ccagacccccgcccatcccctccccgccctgatgtgtgcatggctgaccccgaggggctcagctcagagtctgggagagtagagaggctacgggagaaggaaaaggttcaggggcgagtagggcgcagggccccaggcaaggccaagccagcgtcccctgcacggcgtctggatcttcggggaaaacgctcacccacccctggtaaagggcctgcagatcgagcatcccgggccccacctcgaccacgcagcaccacaagccaggtcaccccagcagaggaaaaggatggacacagccccatgtccaaaggcctagtcaatggactcaaggcaggaccaatggccttgagttccaagggcagctctggtgcccctgtatatgtggatctcgcctacatcccgaatcattgcagtggcaagactgctgaccttgacttcttccgtcgagtgcgtgcatcctactatgtggtcagtgggaatgaccctgccaatggcgagccaagccgggctgtgctggatgccctgctggagggcaaggcccagtggggggagaatcttcaggtgaaggtgactctgatccctactcatgacacggaggtgactcgtgagtggtaccaacaaactcatgagcagcagcaacaactgaatgtcctggtcctggctagcagcagcaccgtggtgatgcaggatgagtccttccctgcctgcaagattgagttctga

(19) United States
(12) Patent Application Publication
(10) Pub. No.: US 2004/0210042 A1
(43) Pub. Date: Oct. 21, 2004

(54) Polypeptides relating to signal transfer of advanced glycation end product receptor

(76) Inventor: Jun-ichi Tsuchida, Tokyo (JP)

Correspondence Address: LEYDIG VOIT & MAYER, LTD, TWO PRUDENTIAL PLAZA, SUITE 4900, 180 NORTH STETSON AVENUE, CHICAGO, IL, 60601-6780, US

(21) Appl. No.: 10/484,364

(22) PCT Filed: Jul. 18, 2002
(86) PCT No.: PCT/JP02/07344

(30) Foreign Application Priority Data
Jul. 19, 2001 (JP) 2001-219122

(51) Int. Cl. C07K 16/28

(52) U.S. Cl. 530/388.22

(57) ABSTRACT

It is intended to provide polypeptides which directly or indirectly bind to a cytoplasmic domain of RAGE and inhibits the signal transduction from binding of a ligand to RAGE (the receptor for advanced glycation endproduct) through activation of NFκB. Typical examples thereof are polypeptides respectively having amino acid sequences represented by SEQ ID NOS:1 to 32, SEQ ID NOS:67 to 79 and SEQ ID NOS:80 to 86 in Sequence Listing or the amino acid sequence including deletion, substitution or addition of one to several amino acids. Also, polypeptides involved in the signal transduction of RAGE are provided. Moreover, a pharmaceutical for gene therapy using polynucleotides encoding these polypeptides: a method for screening a useful compound using these polypeptides: and pharmaceutical composition and diagnostic reagent are also provided.

### TECHNICAL FIELD

The present invention relates to novel polypeptides. In particular, the present invention relates to novel polypeptides involved in a signal transduction of the receptor for an advanced glycation end product (AGE) (hereinafter, the receptor is also referred to as “RAGE”) and a polynucleotide encoding the same.

### BACKGROUND ART

A non-enzymatic reaction of glucose or another reducing sugar with an amino group in a protein or in a lipid generates a Schiff base, and then an irreversible reaction thereof results in advanced glycation endproducts (AGE). AGE is produced and accumulated by aging or hyperglycemia, and it cause denaturation of proteins, production of active oxygen, and activation of cells and inflammation. The accumulation of AGE is suggested in various kinds of diseases including diabetes, Alzheimer's Disease, dialysis amyloidosis, and also in natural aging.

A binding to a specific receptor is regarded as one of the mechanisms of AGE's action. Several receptors for AGE are known. Among them, RAGE (the receptor for AGE: AGE receptor) is considered to be a main receptor. RAGE is a single transmembrane receptor cloned in 1992, having three immunoglobulin domains on the extracellular domain. Examples of ligands thereof include AGE, amphoterin, and β-amyloid.

It is known in the art that the binding of AGE to RAGE causes activation of NFκB by an oxidative stress. However, a signal transduction from RAGE through NFκB has not been revealed up to now.

If a molecule involved in such a pathway is identified, it may be possible to block a signal transduction of RAGE and thereby, inhibit the onset of a disease such as diabetes, a diabetic complication, Alzheimer's Disease, dialysis amyloidosis, a cancer, a periodontal disease, or aging, by (i) exogenously introducing the molecule, (ii) inhibiting the expression of the molecule by a small molecule, (iii) inhibiting the function of the molecule by a small molecule, or the like,

### DISCLOSURE OF THE INVENTION

That is, an object of the present invention is to obtain a molecule involved in a signal transduction of RAGE and to use the molecule for treatment of diseases such as those listed above or for screening of pharmaceuticals. In particular, (i) a gene therapy in which the obtained gene is introduced into human body, (ii) screening or evaluation of pharmaceuticals in cells in which the obtained gene is introduced, or the like can be exemplified.

For finding out a protein to interact with a cytoplasmic domain of RAGE, the inventors of the present invention have screened human kidney cDNA libraries using a yeast two-hybrid system. Consequently, 31 polynucleotides predicted to bind to a cytoplasmic domain of RAGE have been obtained. Furthermore, seven polynucleotides have been obtained as a result of screening human brain cDNA libraries, and thus the present invention has been completed.

That is, a gist of the present invention resides in a polypeptide, which is a substantially purified polypeptide, and is characterized by:

(a) directly or indirectly binds to a cytoplasmic domain of receptor for an advanced glycation end product (AGE); and

(b) inhibits a signal transduction from binding of a ligand to receptor for the advanced glycation endproduct (AGE) through activation of NFκB.

According to a preferable embodiment of the above, there can be mentioned an amino acid sequence selected from SEQ ID NOS: 1 to 32, SEQ ID NOS: 67 to 79, and SEQ ID NOS: 80 to 86 in the Sequence Listing or the amino acid sequence including deletion, substitution, or addition of one or several amino acids. According to a further preferable embodiment of the present invention, there can be mentioned an amino acid sequence selected from SEQ ID NOS: 11, 12, 29, and 30 in the Sequence Listing or the amino acid sequence including deletion, substitution, or addition of one or several amino acids.

As a second gist of the present invention, a polynucleotide which encodes the above-described polypeptide can be mentioned. According to a preferable embodiment thereof, there can be mentioned a nucleotide sequence represented by one of SEQ ID NOS: 33 to 63 and SEQ ID NOS: 87 to 93 or a nucleotide sequence hybridizable with the polynucleotide represented by the nucleotide sequence under a stringent condition. According to a further preferable embodiment, there can be mentioned a nucleotide sequence represented by one of SEQ ID NOS: 43, 44, 61, and 62 or a nucleotide sequence hybridizable with the polynucleotide represented by the nucleotide sequence under a stringent condition.

Further, as a third gist of the present invention, there can be mentioned a pharmaceutical for a gene therapy, comprising a vector which is expressible in an animal and contains the above-described polynucleotide. According to a preferable embodiment thereof, there can be mentioned a pharmaceutical for treating diabetes, a diabetic complication, Alzheimer's Disease, dialysis amyloidosis, cancer, periodontal disease, or an aging-related disease.

Further, as another gist of the present invention, there can be mentioned a vector containing the above-mentioned polynucleotide; a microorganism or cell which is transformed with the vector; and a method for producing the polypeptide, comprising culturing the microorganism or cell and isolating the polypeptide from the culture.

As a fourth gist of the present invention, there can be mentioned a method for screening a substance that inhibits or accelerates the biding of the above-mentioned polypeptide to a cytoplasmic domain of the receptor for AGE, comprising placing a target of screening in a screening system that contains a cytoplasmic domain of the receptor for AGE and the polypeptide and measuring a degree of inhibition or acceleration of the binding of the polypeptide to the cytoplasmic domain of the receptor for AGE.

As a screening method, there can be mentioned as another gist of the present invention, a method of screening a substance that enhances or inhibits a function of the polypeptide or a substance that increases or decreases an amount of the polypeptide, comprising placing a target of screening in a screening system that contains the above-mentioned polypeptides and measuring a degree of enhancement or inhibition of the function of the polypeptide or a degree of increase or decrease in the amount of the polypeptide.

As a fifth gist of the present invention, there can be mentioned a compound which is obtainable by the screening method described above, and according to a preferable embodiment thereof, there can be mentioned a compound characterized by:

(a) directly or indirectly binds to a cytoplasmic domain of the receptor for an advanced glycation endproduct (AGE); and

(b) inhibits a signal transduction from binding of a ligand with the receptor for advanced glycation endproduct (AGE) through activation of NFκB.

Further, as another gist of the present invention, there can be mentioned a pharmaceutical composition comprising, as an effective ingredient, the substance selected from the group consisting of the above-mentioned compound, a salt thereof, a hydrate thereof, and a solvate thereof. According to a preferable embodiment, the pharmaceutical composition may be used for treating a disease selected from diabetes, a diabetic complication, Alzheimer's Disease, dialysis amyloidosis, a cancer, a periodontal disease, and an aging-related disease.

Further, as another gist of the present invention, there can be mentioned an antibody which can specifically bind to the polypeptide; a polynucleotide which is represented by a nucleotide sequence having at least 20 sequential nucleotides in any of the sequences of SEQ ID NOS: 33 to 63 and SEQ ID NOS: 87 to 93, preferably in any of the sequences of SEQ ID NOS: 43, 44, 61, and 62 in the Sequence Listing, a sequence complementary to the nucleotide sequence, and a nucleotide sequence hybridizable with the nucleotide sequence under a stringent condition; a probe comprising the polynucleotide and a label; and a diagnostic reagent including the probe. According to a preferable embodiment, the diagnostic reagent may be used for a diagnosis of diabetes, a diabetic complication, Alzheimer's Disease, dialysis amyloidosis, a cancer, a periodontal disease, and an aging-related disease.

### BRIEF DESCRIPTION OF THE DRAWING

FIG. 1 is a diagram that illustrates functions of the cloned polypeptides.

### BEST MODE FOR CARRYING OUT THE INVENTION

Hereinafter, the present invention is described specifically. The polypeptide of the present invention is a substantially pure polypeptide which can directly or indirectly bind to a cytoplasmic domain of RAGE and inhibit signal transduction from binding of a ligand to RAGE through activation of NFκB.

The “direct biding” referred to herein means a direct binding of the polypeptide of the present invention to a cytoplasmic domain of RAGE, and the “indirect biding” means a binding of the polypeptide of the present invention to a cytoplasmic domain of RAGE through a protein or the like preexisting in an environment where the polypeptide of the present invention can bind to a cytoplasmic domain of RAGE (in an assay system or a human body, etc.). As “ligand”, there can be mentioned AGE, amphoterin, β-amyloid, or the like. For instance, when the polypeptide of the present invention is used for a method of screening pharmaceuticals or for a gene therapy which will be described below, the ligand can be selected as appropriate depending on a target disease. For example, AGE can be selected when the target disease is diabetes or a diabetic complication, amphoterin can be selected when the target disease is a cancer, and β-amyloid can be selected when the target disease is Alzheimer's Disease.

In the present invention, the “polypeptides” includes those typically recognized as peptides, oligopeptides, polypeptides, and proteins in the art. Natural proteins and chemically-synthesized or recombinantly-engineered polypeptides and peptides are also included. The polypeptide may or may not be subjected to a post-translational modification such as glycosylation or phosphorylation.

A precursor of the polypeptide of the present invention is also included in the polypeptide of the present invention as far as it has the physiological activities as described above. Examples of such a precursor include one having one or more amino acids added to the N-terminus and (or) the C-terminus of the peptide of the present invention.

Furthermore, the polypeptide of the present invention may be bound to polyethylene glycol for prolonging the half-period thereof, or fused with a secretory sequence or a leader sequence for secretion. Alternatively, the polypeptide of the present invention may be produced as a fusion polypeptide for purification.

A fragment characterized by the structural or functional properties of the polypeptide of the present invention is also useful.

A physiologically acceptable acid-added salt of the polypeptide of the present invention or of a precursor of the polypeptide is also included in the present invention. As a acid-added salt, there can be mentioned a salt with an inorganic acid such as hydrochloric acid, phosphoric acid, or sulfuric acid; and a salt with an organic acid such as acetic acid, formic acid, fumaric acid, maleic acid, succinic acid, citric acid, tartaric acid, malic acid, benzoic acid, or benzenesulfonic acid.

The “amino acid sequence including deletion, substitution or deletion of one or several amino acids” of the present invention means an amino acid sequence of a mutant of an allelic mutation or a mutation found in nature, or an artificial mutation or a mutation obtainable by using recombinant technology. All of the amino acid sequences to be included in the present invention are polypeptides having the same activity as that of the novel polypeptide molecule of the present invention. Even if only one amino acid residue is modified, an amino acid sequence including variation which causes the loss of the activity is not included in the present invention.

The “polynucleotide” of the present invention is a polypeptide that encodes the polypeptide of the present invention as described above. Specifically, the polynucleotide of the present invention is a polynucleotide encoding an amino acid sequence represented by one of SEQ ID NOS: 1 to 32, SEQ ID NOS: 67 to 79, and SEQ ID NOS: 80 to 86, preferably one of SEQ ID NOS: 11, 12, 29, and 30 in the Sequence Listing. Specifically, such a polynucleotide can be exemplified by a polynucleotide having a nucleotide sequence represented by one of SEQ ID NOS: 33 to 63 and SEQ ID NOS: 87 to 93, preferably one of SEQ ID NOS: 43, 44, 61, and 62. However, as far as the same amino acid sequence is encoded, each codon may be substituted with other equivalent codon.

The polynucleotide of the present invention may be a polynucleotide encoding a polypeptide having substantially the same amino acid sequence as the amino acid sequence represented by one of SEQ ID NOS: 1 to 32, SEQ ID NOS: 67 to 79, and SEQ ID NOS: 80 to 86, preferably one of SEQ ID NOS: 11, 12, 29, and 30. Such a polynucleotide is preferably a polynucleotide having at least 80% homology to the nucleotide sequence represented by one of SEQ ID NOS: 33 to 63 and SEQ ID NOS: 87 to 93, preferably one of SEQ ID NOS: 43, 44, 61, and 62, or its complementary nucleotide sequence. A polynucleotide having 90% homology is more preferable, and in particular, the most preferable is a polynucleotide having 95% or more homology. As a polynucleotide having 95% or more, preferably 97% or more homology, there can be mentioned a polynucleotide hybridizable with the polynucleotide represented by one of SEQ ID NOS: 33 to 63 and SEQ ID NOS: 87 to 93, preferably one of SEQ ID NOS: 43, 44, 61, and 62 or a probe prepared from the sequence under a stringent condition.

Furthermore, the present invention includes a polynucleotide that encodes a fragment characterized by the structural or functional properties of the polypeptide of the present invention. Such a polynucleotide fragment and a polynucleotide hybridizable with a polynucleotide that encodes the fragment are useful as PCR primers as well as probes for detecting a DNA that encodes the peptide of the present invention.

Hereinafter, the characteristic features included in the present invention are described with reference to Examples described below as a representative example. However, the present invention is not limited to these examples.

For the purpose of obtaining a novel molecule involved in the signal transduction from binding of AGE to RAGE through activation of NFκB, a gene for a cytoplasmic domain of RAGE was amplified by a PCR method, as shown in Example 1 described later. Then, using the gene for a cytoplasmic domain of RAGE, human kidney cDNA libraries and brain cDNA libraries were screened by a yeast two-hybrid method using the LexA system. Consequently, so far as the inventor of the present invention knows, a polynucleotide was obtained, which encodes a novel polypeptide having no homology to any protein that has been reported so far.

In this way, 31 polynucleotides and 7 polynucleotides predicted to bind to a cytoplasmic domain of RAGE were obtained. Their nucleotide sequences are represented by one of SEQ ID NOS: 33 to 63 and 87 to 93, preferably one of SEQ ID NOS: 43, 44, 61, and 62 in the Sequence Listing.

The nucleotide sequence of the polynucleotide cloned as described above can be linked with a vector which is expressible in a host cell and contains transcriptional control activities such as a promoter, an operator, or an enhancer; a termination sequence; and other regulatory sequences for controlling the expression of RAGE.

A recombinant vector containing the polynucleotide of the present invention is used for the production of the polypeptide of the present invention. The vector of the present inventionto be used is one suitable for retention, amplification, and expression of a polynucleotide in a host cell. The cloned polynucleotide that encodes the polypeptide of the present invention can be inserted into a vector directly or after being digested with a restriction enzyme or after being linked to a linker. A DNA has a translation initiation codon (ATG) on its 5′-terminal side and a translation termination codon (TAA, TGA, or TAG) on its 3′-terminal side. A DNA is located downstream from a promoter in the expression vector.

Examples of such a vector include: plasmids derived from Escherichia coli (such as pBR322, pBR325, and pUC12), plasmids derived from Bacillus subtilis (such as pUB110 and pC194), plasmids derived from the genus Streptomyces, and plasmids derived from the genus Salmonella; plasmids derived from yeast episomes and host chromosome elements (such as YCp plasmids and PYAC plasmids), bacteriophages such as λ-phage and vectors derived from viruses such as Vaccinia virus, Adenovirus, Retrovirus, and Baculovirus. Many of those vectors are commercially available.

A DNA sequence in the recombinant vector is operably-linked to an appropriate expression control sequence (promoter). Examples of such a promoter include: phage λPL promoter and T7 promoter; Escherichia coli lac, trp, 1pp, and c promoters; Bacillus subtilis SPO1 promoter and penP promoter; yeast PHO5 promoter, PGK promoter, GAP promoter, ADH1 promoter, SUC2 promoter, GAL4 promoter, and MFα promoter; insect cell polyhedron promoter and P10 promoter; and animal cell SV40 early or late promoter, retrovirus LTR promoter, CMV promoter, HSV-TK promoter, and metallothionein promoter.

In general, the expression vector contains an expression control region to be regulated by a repressor-binding region, an enhancer, or the like. In addition, the expression vector contains a selection marker. Examples of appropriate markers include a dihydrofolate reductase (dhfr) gene and a neomycin-resistance gene for eukaryotic cells, and a tetracycline or ampicillin resistance gene for bacterial cells. The dhfr gene provides a transformed cell with methotrexate resistance, while the neomycin-resistance gene provides a transformed cell with G418 resistance. When a host cell is a dhfr gene-deficient CHO cell and the dhfr gene is used as a selection marker, a transformant can be selected in a thymidine-free medium. In this case, a resistant strain can be selected by culturing cells under a gradually increasing concentration of methotrexate (MTX). Thus, a DNA that encodes the peptide of the present invention is amplified in the cell simultaneously with the amplification of the dhfr gene, thereby a CHO (dhfr-) cell with high level expression can be obtained.

The recombinant vector of the present invention is constructed in a way that a signal sequence is added to the N terminus of the peptide, if required. Such a signal sequence may be: a PhoA or OmpA signal sequence or the like in the case of an Escherichia coli host; an Mfα or SUC2 signal sequence or the like in the case of a yeast host; and an α-interferon signal sequence or the like in the case of an animal host.

The present invention also relates to a host cell that harbors the recombinant vector as described above. Examples of the host cells include mammalian cells, plant cells, insect cells, yeast cells, eukaryotic cells such as Aspergius fungi, and prokaryotic cells such bacterial cells. The recombinant vector of the present invention can be introduced into the host cell by means of calcium-phosphate transfection, electroporation, transduction, infection, or the like.

Under the control of the promoter as described above, the peptide of the present invention can be expressed in hosts such as mammalian cells, yeasts, and bacteria as described above.

Examples of a prokaryotic host include Escherichia coli, Bacillus subtilis, Salmonella, Pseudomonas, Streptomyces, and Staphylococcus. Examples of the yeasts include Saccharomyces cerevisiae, Schizosaccharomyces pombe, and Pichia pastoris.

The transformed prokaryotic host is proliferated, and in a case of using a vector containing an inducible promoter, a temperature or a chemical inducer can initiate the induction. Then, the cells are cultured in a liquid medium containing a carbon source (such as glucose, dextran, or soluble starch), a nitrogen source (such as ammonium salt, nitrate salt, peptone, casein, meat extract, or bean cake), and an inorganic substance (such as calcium chloride, sodium dihydrogen phosphate, or magnesium chloride) at a suitable pH (i.e., at a pH of about 5 to 8) for a suitable period (i.e., for about 3 to 24 hours). The suitable culture temperature is about 14 to 43° C. for Escherichia coli and about 30 to 40° C. for bacteria of the genus Bacillus. The cell is physically or chemically disrupted after cultivation, and then the peptide of the present invention is purified from the resulting crude extract.

The transformed yeast is cultured in a medium such as a minimal medium at a pH of about 5 to 8 and at a temperature of about 20 to 35° C. for about 24 to 72 hours.

Insect cells to be used are Sf cells, MG1 cells, or the like when the virus is AcNPV (Autographa californica NPV), larval silkworms, silkworm cultured cells (BM-N cells) or the like when the virus is Bombyx polynucleosis virus (BmPV). For the silkworm cells, a medium such as a TC-10 medium containing a heat-inactivated 10% fetal bovine serum is used, and the cells are cultured to be confluent at about 27° C., then the cells are subjected to passage.

Examples of the mammalian cell include COS-7 cell, mouse AtT-20 cell, rat GH3 cell, rat MtT cell, mouse MIN6 cell, Vero cell, C127 cell, CHO cell, dhfr-gene defect CHO cell, HeLa cell, L cell, BHK cell, BALB3T3 cell, 293 cell, Bowes melanoma cell and the like.

Expression vectors for mammalian cell include a replication origin, a promoter (such as the above-mentioned SV40 early or late promoter, retrovirus LTR promoter, CMV promoter, HSV-TK promoter, or metallothionein promoter), an enhancer (such as SV40 enhancer, adenovirus enhancer, or cytomegalovirus early promoter), a selection marker (such as the above-mentioned dhfr gene or neomycin-resistance gene), a libosome binding site, a polyadenylation site (such as SV40 polyadenylation site), splice donor and acceptor sites (such as a DNA sequence derived from SV40 splice site), a transcription termination sequence, and a 5′-non-transcription sequence.

As such a vector, a plasmid vector, a single- or double-stranded phage vector, a single- or double-stranded RNA or DNA virus vector, or the like can be used. As a medium for the transformed mammalian cells, an MEM medium, a DMEM medium, an RPMI-1640 medium, or the like, which contains a fetal bovine serum at a concentration of about 5 to 20%, can be used. The culture of the cells is performed at a pH of about 6 to 8 and a temperature of 30 to 40° C. for about 15 to 72 hours.

When the mammalian cells are used as hosts, the polypeptide of the present invention can be collected and purified from a culture of the recombinant cells using ammonium sulfate or ethanol precipitation, acid extraction, anion- or cation-exchange chromatography, hydrophobic interaction chromatography, or the like.

The polypeptide of the present invention may not be glycosylated. In addition, depending on hosts, a polypeptide having methionine at its N-terminus can be obtained.

Furthermore, the polynucleotide of the present invention is useful in a gene therapy for treating a disease such as diabetes, a diabetic complication, Alzheimer's Disease, dialysis amyloidosis, a cancer, a periodontal disease, or an aging-related disease. The “gene therapy” means the administration of a gene or gene-incorporated cells into the human body for treating a disease. For a pharmaceutical use, any of a purified polynucleotide, a recombinant, a culture liquor of a transformant, an isolated transformant, a treated-transformant, a fixed-transformant, a crude enzyme solution, an enzyme-treated product, etc. may be used.

When the polynucleotide of the present invention is used for a gene therapy, various technologies conventionally used for a gene therapy can be employed. Specifically, a gene containing the polynucleotide of the present invention can be expressed in the body by implanting the expression vector for the polynucleotide of the present invention, which is obtained from a virus vector such as a retrovirus vector, an adenovirus vector, an AAV vector, or a herpes virus vector or which is obtained by a membrane-fusion liposome method, into bone marrow cells of a patient suffering from a disease such as diabetes, a diabetic complication, Alzheimer's Disease, dialysis amyloidosis, a cancer, a periodontal disease, or an aging-related disease (hereinafter, which may be collectively referred to as “the disease”), according to a method such as one described in JP 09-501046 A or a method based thereon; by administering the expression vector into the muscle tissue, blood system, intestine, lung, or the like of a patient suffering from the disease according to a method such as one described in JP 09-505084 A or a method based thereon; or by administering the expression vector into the cerebrospinal fluid of a patient suffering from the disease according to a method such as one described in JP 09-505561 A or a method based thereon. Furthermore, an offspring of the patient can be prevented from suffering from the disease by incorporating the gene containing the polynucleotide of the present invention into the egg cell of the patient suffering from the disease.

The polypeptide or polynucleotide of the present invention is useful for identifying a compound which can modulate control of the signal transduction from binding of a ligand to RAGE through activation of NFκB. That is, the present invention provides a method of screening a substance that inhibits or accelerates a binding of the polypeptide of the present invention to a cytoplasmic domain of RAGE. The method includes: placing a target of screening in a screening system containing a cytoplasmic domain of RAGE and the polypeptide of the present invention; and measuring the degree of inhibition or acceleration of the binding of the polypeptide of the present invention to a cytoplasmic domain of RAGE.

In the present invention, the “target of screening” is not particularly limited as far as it is a substance available for a screening, the target may be a high molecule as well as a low molecule. For instance, the target of screening may be a peptide, an analog thereof, a microbial culture liquor, or an organic compound. In the present invention, “screening” is used in the meaning of including an assay.

The screening system is not limited as far as it is a method typically used in the art, for instance, an experimental system such as a yeast two-hybrid system can be used. That is, the interaction between the polypeptide of the present invention and a cytoplasmic domain of RAGE is monitored by the yeast two-hybrid activity, and then a substance that inhibits or enhances the reporter activity thereof may be screed. In an alternative method, using the BIAcore (manufactured by BIACORE K.K.), a change in their interaction is monitored in the presence of a substance to be provided as a screening target to identify the influence of the substance on their interaction. In the screening described herein, not only a polypeptide having the full-length polypeptide of the present invention having an amino acid sequence selected from SEQ ID NOS: 1 to 32, SEQ ID NOS: 67 to 79, and SEQ ID NOS: 80 to 86 in the Sequence Listing but also a partial peptide having at least five amino acid residues can be used as far as the target substance can be screened in the above method. Preferably, it can be also possible to use not only a polypeptide having the full-length polypeptide of the present invention having an amino acid sequence selected from SEQ ID NOS: 11, 12, 29, and 30 but also a partial polypeptide having at least five residues as far as the target substance can be screened in the above method.

In the present invention, “measure the degree of inhibition of binding” is used in the meaning of including measuring the presence or absence of the binding.

The methods as described above are methods in which a cytoplasmic domain of RAGE is provided in the screening system. However, in the present invention, it is also possible to perform a method in which a cytoplasmic domain of RAGE is not provided in a screening system. That is, there is provided a method of screening a substance that enhances or inhibits the function of the polypeptide of the present invention or a substance that increases or decreases an amount of the polypeptide of the present invention, which method comprising: placing a target substance in a screening system containing the polypeptide of the present invention; and measuring the degree of enhancement or inhibition of the function of the polypeptide of the present invention or measuring the degree of an increase or decrease in an amount of the polypeptide of the present invention.

The screening target, the definition of the screening, and the screening system are as described above. “Measuring the degree of enhancement or inhibition of the function” and “measuring the degree of an increase or decrease in an amount” are used in the meaning of measuring the presence or absence of enhancement or inhibition of the function and measuring the presence or absence of an increase or decrease in the amount, respectively.

The screening system is not limited as far as it is a method conventionally used in the art, for instance, a vector containing the polypeptide of the present invention is introduced into a cell in which pNFk-luc (STRATAGENE Co., Ltd.) is introduced; and then, a substance that enhances or inhibits the NFκB activity is screened.

As described above, the polypeptide of the present invention is involved in the signal transduction from RAGE through NFκB, so that the screening method of the present invention can be used as a method of screening a substance useful as a preventive agent and/or a therapeutic agent for treating a disease such as diabetes, a diabetic complication, Alzheimer's Disease, dialysis amyloidosis, a cancer, a periodontal disease, or an aging-related disease.

The compounds obtainable by using the screening method of the present invention and the salts thereof, and their hydrates and solvates are compounds that modify the binding of the polypeptide of the present invention to a cytoplasmic domain of RAGE (i.e., the compounds inhibit or accelerate the binding) or compounds having the cell-stimulating activities caused by the interaction between the polypeptide of the present invention and a cytoplasmic domain of RAGE. Examples of the compounds include peptides, proteins, fermented products, non-peptide compounds, and synthetic compounds. These compounds may be novel or known compounds.

As a pharmaceutical composition, for example, the compound itself may be administered alone. It is preferable to prepare a pharmaceutical composition containing the above compound as an effective ingredient by using a pharmaceutically acceptable pharmaceutical additive, and administrate the composition. The pharmaceutical composition may be orally or parenterally administered in a form of a pharmaceutical composition or preparation (for example, a tablet, a pill agent, a capsule, a granule, powder, syrup, an emulsion agent, an elixir agent, a suspending agent, a resolvent, an injection, a drop, or a suppository) which is obtainable by mixing the compound of the present invention with a pharmaceutically acceptable carrier (for example, an excipient, a binder, a disintegrating agent, a flavoring substance, a deodorizing substance, an emulsifier, an attenuant, or a solubilizer). The pharmaceutical composition can be manufactured according to the conventional method. In this specification, “parenterally” covers a hypodermic injection, an intravenous injection, an intramuscular injection, an intraperitoneal injection, and a drip method. A composition for injection can be prepared by the method known in the art. A rectal suppository can be produced by mixing the pharmaceutical compound with a suitable excipient. Examples of solid formulation for oral administration include those as described above such as powder, a granule, a tablet, a pill agent, and a capsule. Examples of liquid formulations for oral administration include an emulsion agent, syrup, an elixir agent, a suspending agent, and a solution agent, each of which is acceptable as a medicine.

Furthermore, each of the preparations as described above can be prepared by conventional procedures. The clinical dosage of the pharmaceutical of the present invention is determined by appropriately increasing or decreasing on the basis of the substance used as an effective ingredient, age, condition, symptom, the presence or absence of simultaneous administration, and soon. The above-described dosage per day may be administered once a day or two or several times per day with appropriate intervals. Alternatively, the dosage may be intermittently administered every several days.

On the basis of the nucleotide sequence information about the polynucleotide of the present invention, it is possible to obtain an antisence specific to a polynucleotide complementary to the sequence. The “antisence” referred to herein means a polynucleotide complementary to at least a part of mRNA or DNA that encodes the polynucleotide of the present invention, which inhibits the transcription and translation of the polynucleotide of the present invention. Furthermore, using the transformant of the present invention, the effects of the antisence can be also confirmed. For such an antisense, there can be used not only typical DNAs and RNAs but also any other sequence-specific nucleotide-like molecule considered to have antisence effects.

The polynucleotide and polypeptide of the present invention can be used for diagnosing the above-described diseases. An antibody specific to the polypeptide of the present invention can be obtained by using proteins which is produced in large amounts in a cell transformed by the polynucleotide of the present invention. As an immunizing antigen, the full-length polypeptide of the present invention or a peptide fragment consisting of at least five sequential amino acid residues of the amino acid sequence of the polypeptide of the present invention may be used. In the present invention, the antibodies include, in addition to the complete antibodies, all of the molecules which are produced from original antibody and considered to have antibody effects, for example, Fab, F(ab′)2, Fv, and scFv fragments that contain the respective antigen-binding sites.

Using the antibodies as described above, it is possible to construct the system of ELISA or RIA or the system of western blotting for detecting the polypeptide of the present invention in cells and tissues. For instance, such a detecting system can be used for diagnosing the diseases as described in the explanation of the screening method.

Furthermore, in the present invention, the expression of the polypeptide of the present invention in cells or tissues can be detected using a probe that contains a suitable label and the polynucleotide represented by a nucleotide sequence comprising at least 20 sequential nucleotides in the sequence of any of SEQ ID NOS: 33 to 63 and SEQ ID NOS: 87 to 93, preferably any of SEQ ID NOS: 43, 44, 61, and 62 in the Sequence Listing, a complementary sequence thereof, and a nucleotide sequence hybridizable with the nucleotide sequence under a stringent condition. Therefore, such a probe can be also used in the assay for diagnosis. A label used in this case is not particularly limited as far as it can be used in the conventional assay for the diagnosis. As such a label, for example, avidin or biotin, an enzyme such as peroxidase, a radioactive isotope, a fluorescent substance, or an antigen can be used. Using those labels, the nucleotide sequences are labeled by a method conventionally used in the art, and the labels can be detected by suitable methods, respectively.

### EXAMPLES

Hereinafter, the present invention will be described in more detail with reference to the examples. However, the present invention is not limited only to these examples.

### Example 1

Cloning of a Protein Which Interacts With a Cytoplasmic Domain of RAGE by Yeast Two-Hybrid Method

A gene for a cytoplasmic domain of RAGE (SEQ ID NO: 64 in the Sequence Listing) (J. Biol. Chem Jul. 25, 1992 267 (21): 14998-5004) was amplified using synthetic oligonucleotides: 5′-ACGTGAATTCAGGCGGCAACGCCGAGGAG-3′ (SEQ ID NO: 65 in the Sequence Listing) and 5′-CGATCTCGAGTCAAGGCCCTCCAGTACTACTC-3′ (SEQ ID NO: 66 in the Sequence Listing), and was then digested with restriction enzymes EcoRI and XhoI. Also, a vector pHybLex/Zeo (manufactured by Invitrogen Co., Ltd.) was digested with the restriction enzymes EcoRI and XhoI. Subsequently, the both genes were subjected to a 14-hour ligation reaction using Takara Ligation Kit ver. 2 (manufactured by Takara Shuzo Co., Ltd.) and used for transforming E.coli strain DH5α (manufactured by Takara Shuzo Co., Ltd.), thereby colonies were obtained. Consequently, a vector (pHybLex/Zeo-RAGECD) in which the cytoplasmic domain of RAGE is incorporated into the pHybLex/Zeo was obtained. Using a DNA sequencer (manufactured by Beckman Co., Ltd.), the sequence of the cytoplasmic domain of RAGE was confirmed.

The vector pHybLex/Zeo-RAGECD obtained as described above was introduced into Yeast L40 strain attached to the MATCH MAKER kit which is described below, and then, a clone into which the pHybLex/Zeo-RAGECD had been introduced was selected.

Then, the Human kidney MATCHMAKER LexA library (manufactured by Clontech Co., Ltd.) was introduced to the above-described strain and 10⁶ colonies were screened. At the first screening, a histidine-deficient medium was used and about 200 colonies were selected based on the expression of a histidine gene, which was a reporter of the interaction. At the second screening, 31 colonies were obtained based on the expression of LacZ, which was another reporter of the interaction. Finally, 31 clones each of which had been confirmed to surely have two kinds of reporter activities were selected. The above procedures were carried out on the basis of protocols of the yeast two-hybrid method from Invitrogen Co., Ltd. and Clontech Co., Ltd.

The library vectors were recovered from the yeast colonies selected as described above, and the nucleotide sequences were analyzed using the DNA sequencer (the same as above). The nucleotide sequences are shown in SEQ ID NOS: 33 to 63 in the Sequence Listing.

The polypeptide obtained as described above (in a state of being inserted in the pB42AD vector (manufactured by Clontech Co., Ltd.) by the screening) was excised from the vector with the restriction enzymes EcoRI and XhoI. Then, the resulting fragments were subjected to a ligation reaction, using the Ligation Kit ver. 2 (manufactured by Takara Shuzo Co., Ltd.), with the pGBKT7 vector (manufactured by Clontech Co., Ltd.) which had been digested with the restriction enzymes EcoRI and SalI, thereby, the polypeptide-incorporated pGBKT7 vector was obtained. Similarly, the RAGECD fragments were excised from the above-described pHybLex/Zeo-RAGECD vector with the restriction enzymes EcoRI and XhoI, followed by being ligated to a pGADGH vector (manufactured by Clontech Co., Ltd.) which had been digested with the restriction enzymes EcoRI and SalI, thereby a pGBKT7-RAGECD vector was obtained.

The pGBKT7-polypeptide vector was introduced into a yeast strain AH109 (manufactured by Clontech Co., Ltd.), and the pGADT7-RAGECD vector was introduced into a yeast strain Y187 (manufactured by Clontech Co., Ltd.), by using the lithium acetate method (see page 20 of “Yeast Protocols Handbook” attached to the kit of Clontech Co., Ltd.).

The plasmid-introduced yeasts were subjected to selection on a tryptophan-deficient SD medium or on a leucine-deficient SD medium, and selected strains were then conjugated with the following combinations (see page 44 of “Yeast Protocols Handbook” attached to the kit of Clontech Co., Ltd.). Subsequently, the yeast in which two kinds of plasmids had been introduced was selected.

1. pGBKT7-polynucleotide of SEQ ID NO: 43+pGADT7-idle vector

2. pGBKT7-polynucleotide of SEQ ID NO: 44+pGADT7-idle vector

3. pGBKT7-polynucleotide of SEQ ID NO: 61+pGADT7-idle vector

4. pGBKT7-polynucleotide of SEQ ID NO: 62+pGADT7-idle vector

5. pGBKT7-polynucleotide of SEQ ID NO: 43+pGADT7-RAGECD vector

6. pGBKT7-polynucleotide of SEQ ID NO: 44+pGADT7-RAGECD vector

7. pGBKT7-polynucleotide of SEQ ID NO: 61+pGADT7-RAGECD vector

8. pGBKT7-polynucleotide of SEQ ID NO: 62+pGADT7-RAGECD vector

As a result of the growth test of the above strains on the SD medium lacking in tryptophan, leucine, histidine and adenine, strains 1, 2, 3, and 4 were not grown, while strains 5, 6, 7, and 8 were grown.

Therefore, it was shown that the polynucleotides of SEQ ID NOS: 43, 44, 61, and 62 were specifically bind to the RAGECD fragments, respectively.

That is, it was found that the polypeptide translated from each of those polynucleotides binds to the cytoplasmic domain of RAGE.

The above procedures were carried out on the basis of protocols of MATCH MAKER GAL4 Yeast Two Hybrid System 3 (manufactured by Clontech Co., Ltd.).

### Example 2

Cloning of Protein Which Can Interact With a Cytoplasmic Domain of RAGE by Yeast Two-Hybrid Method

A gene for a cytoplasmic domain for RAGE (SEQ ID NO: 65 in the Sequence Listing) (J. Biol. Chem 1992 Jul. 25, 267 (21): 14998-5004) was amplified using synthetic oligonucleotides: 5′-ACGTGAATTCAGGCGGCAACGCCGAGGAG-3′ (SEQ ID NO: 66 in the Sequence Listing) and 5′-CGATCTCGAGTCAAGGCCCTCCAGTACTACTC-3′ (SEQ ID NO: 67 in the Sequence Listing), and was then digested with restriction enzymes EcoRI and XhoI. Also, a vector pGBKT7 (manufactured by Clontech Co., Ltd.) was digested with restriction enzymes EcoRI and Sal I. Subsequently, the both genes were subjected to a 14-hour ligation reaction using the Takara Ligation Kit ver. 2 (manufactured by Takara Shuzo Co., Ltd.) and used to transform E. Coli strain DH5α (manufactured by Takara Shuzo Co., Ltd.), thereby colonies were obtained. Consequently, a vector (pGBKT7-RAGECD) in which the cytoplasmic domain of RAGE is introduced in the pGBKT7 was obtained. Using a DNA sequencer (manufactured by Beckman Co., Ltd.), the sequence of the cytoplasmic domain of RAGE was confirmed.

The vector pGBKT7-RAGECD obtained as described above was introduced into yeast strain AH109 attached to the kit MATCH MAKER described below. Then, a clone into which the PGBKT7-RAGECD had been introduced was selected.

Then, Pretransformated MATCHMAKER library Human Brain (manufactured by Clontech Co., Ltd.) was introduced into the above strain and 10⁶ colonies were screened. At the first screening, a histidine-deficient medium was used, and about 140 colonies were selected based on the expression of a histidine gene, which was a reporter of the interaction. At the second screening, 40 colonies were selected on an adenine-deficient medium based on the expression of adenine, which was another reporter of the interaction.

Finally, 7 clones each of which had been confirmed to surely have two kinds of reporter activities were selected, the library vectors were recovered, and the nucleotide sequences were then analyzed using the DNA sequencer (the same as above). The nucleotide sequences are shown in SEQ ID NOS: 87 to 93 in the Sequence Listing.

The above procedures were carried out on the basis of protocols of the yeast two-hybrid method from Invitrogen Co., Ltd. and Clontech Co., Ltd.

### Example 3

Functional Analysis on the Cloned Polypeptide

Because the polypeptide of SEQ ID NO: 62 was a part of, protein kinase C zeta (PKC zeta), the full-length PKC zeta was cloned in pcDNA 3.1 (+) (manufactured by Invitrogen Co., Ltd.) using the PCR method, and pcDNA 3.1 (+)—PKC zeta vector was obtained.

Using TransIT-LT1 (manufactured by Takara Shuzo Co., Ltd.), pNFkB-luc (manufactured by Stratagene Co., Ltd) was introduced into rat C6 glioma cells (ATCC CCL-107). The cells were cultured in a medium with the addition of geneticin (manufactured by Nacalai Tesque, Inc.) to obtain the drug-resistant cells. Thus, stably expressing cells were obtained.

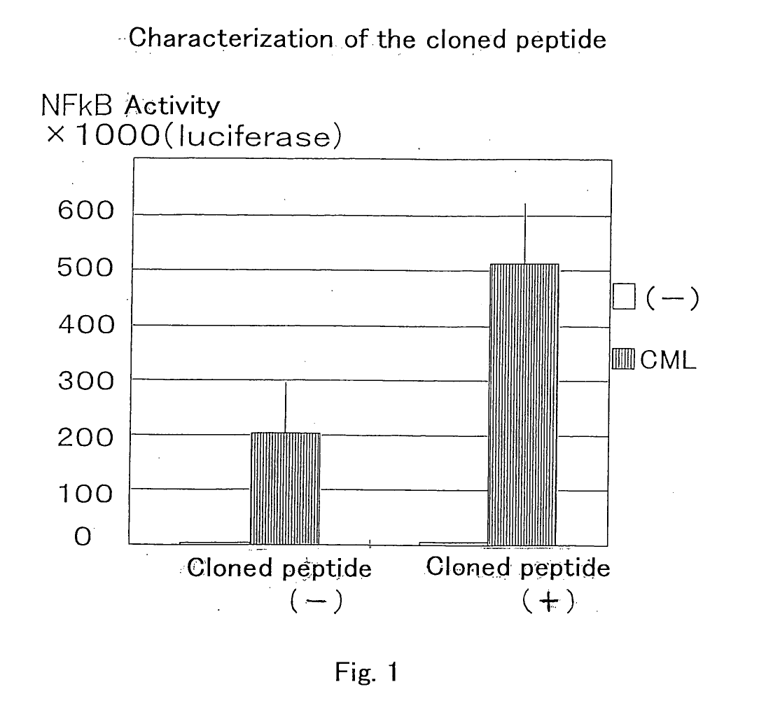

Using the TransIT-LT1 (manufactured by Takara Shuzo Co., Ltd.), pcDNA 3.1 (+) and pcDNA 3.1 (+)—PKC zeta plasmids were introduced into the cells, respectively. After 48 hours, the medium was replaced with a serum-free DMEM medium (manufactured by SIGMA Co., Ltd.). After an additional 12 hours, the cells were stimulated for 6 hours with carboxy methyl lysine (CML) which had been prepared by incubating 1.7 g of BSA (manufactured by SIGMA Co., Ltd.) and 0.36 g of glyoxylic acid (manufactured by OHSIGMA Co., Ltd.) at 36° C. for 24 hours in the presence of a sodium cyano borohydride (manufactured by SIGMA Co., Ltd.) as a catalyst. After the stimulation, a luciferase assay was performed using Bright-Glo (manufactured by Promega Co., Ltd.), in which the cells were measured with an ARVOsx plate reader (manufactured by Wallac Co., Ltd.). Consequently, it was found that the introduction of pcDNA 3.1 (+)—PKC zeta significantly increased the NFkB activity (FIG. 1) as compared to the introduction of an empty vector pcDNA 3.1.

In the case of diseases such as a diabetic complication in which RAGE may be involved, the activation of NFkB by AGE was known in the art. However, the intermediate pathway thereof had been unclear.

From this experiment, it was found that the polypeptide capable of interacting with the intracellular domain of the RAGE increases NFkB activation due to AGE stimulation. Therefore, it was suggested that the polynucleotide of SEQ ID NO: 62 would mediate the downstream signal of RAGE.

Industrial Applicability

According to the present invention, a novel polypeptide involved in the signal transduction of RAGE is obtained. In addition, the polynucleotide encoding the polypeptide of the present invention can be used as a genetic source to be applied in a gene therapy. Furthermore, the polypeptide of the present invention is capable of providing a method of screening a substance useful for treating diseases involved in the signal transduction from RAGE through activation of NFκB, a pharmaceutical composition obtainable by the screening method, and a diagnostic agent for those diseases.

Note that, the present application has been filed claiming a priority of Japanese Patent Application No. 2001-219122.

1. A polypeptide, which is a substantially purified polypeptide, wherein the polypeptide has following characteristics:
(a) the polypeptide directly or indirectly binds to a cytoplasmic domain of the receptor for an advanced glycation endproduct (AGE); and
(b) the polypeptide inhibits a signal transduction from binding of a ligand to the receptor for advanced glycation endproduct (AGE) through activation of NFκB.

2. The polypeptide according to claim 1, which comprises the amino acid sequence selected from SEQ ID NOS: 1 to 32 and SEQ ID NOS: 67 to 79 in the Sequence Listing or said amino acid sequence including deletion, substitution, or addition of one or several amino acids.

3. The polypeptide according to claim 1, which comprises the amino acid sequence selected from SEQ ID NOS: 11, 12, 29, and 30 in the Sequence Listing or said amino acid sequence including deletion, substitution, or addition of one or several amino acids.

4. The polypeptide according to claim 1, which comprises the amino acid sequence selected from SEQ ID NOS: 80 to 86 in the Sequence Listing or said amino acid sequence including deletion, substitution, or addition of one or several amino acids.

5. A polynucleotide, which encodes the polypeptide according to claim 1.

6. The polynucleotide according to claim 5, comprising the nucleotide sequence represented by one selected from SEQ ID NOS: 33 to 63 or a nucleotide sequence which is hybridizable with the polynucleotide represented by one selected from SEQ ID NOS: 33 to 63 under a stringent condition.

7. The polynucleotide according to claim 5, comprising the nucleotide sequence represented by one selected from SEQ ID NOS: 43, 44, 61, and 62 or a nucleotide sequence which is hybridizable with the polynucleotide represented by one selected from SEQ ID NOS: 43, 44, 61, and 62 under a stringent condition.

8. The polynucleotide according to claim 5, comprising the nucleotide sequence represented by one selected from SEQ ID NOS: 87 to 93 or a nucleotide sequence which is hybridizable with the polynucleotide represented by one selected from SEQ ID NOS: 87 to 93 under a stringent condition.

9. A pharmaceutical for a gene therapy, comprising the polynucleotide according to claim 5 and a vector which is expressible in an animal.

10. The pharmaceutical for a gene therapy according to claim 9, which is used for treating a disease selected from diabetes, a diabetic complication, Alzheimer's Disease, dialysis amyloidosis, a cancer, a periodontal disease, and an aging-related disease.

11. A vector, comprising the polynucleotide according to claim 5.

12. A microorganism or cell, which is transformed with the vector according to claim 11.

13. A method for producing a polypeptide comprising:
culturing a microorganism or cell which is transformed with a vector comprising the polynucleotide according to claim 5; and
isolating the polypeptide encoded by the polynucleotide from the culture.

14. A method for screening a substance that inhibits or accelerates the biding of the polypeptide according to claim 1 to a cytoplasmic domain of the receptor for advanced glycation endproduct (AGE), comprising:
placing a target of screening in a screening system that contains the cytoplasmic domain of the receptor for advanced glycation endproduct (AGE) and the polypeptide and
measuring a degree of inhibition or acceleration of the binding of the polypeptide to a cytoplasmic domain of the receptor for advanced glycation end product (AGE).

15. A method for screening a substance that enhances or inhibits a function of the polypeptide according to claim 1 or a substance that increases or decreases an amount of the polypeptide comprising:
placing a target of screening in a screening system that contains the polypeptide and
measuring a degree of enhancement or inhibition of the function of the polypeptide or a degree of increase or decrease of the amount of the polypeptide.

16. A compound, which is obtainable by the screening method according to claim 14.

17. The compound according to claim 16, which has following characteristics:
(a) the compound directly or indirectly binds to a cytoplasmic domain of the receptor for advanced glycation endproduct (AGE); and
(b) the compound inhibits a signal transduction from binding of a ligand to the receptor for advanced glycation endproduct (AGE) through activation of NFκB.

18. A pharmaceutical composition, comprising the substance selected from the group consisting of the compound according to claim 16, a salt thereof, a hydrate thereof, and a solvate thereof.

19. The pharmaceutical composition according to claim 18, which is used for treating a disease selected from diabetes, a diabetic complication, Alzheimer's Disease, dialysis amyloidosis, a cancer, a periodontal disease, and an aging-related disease.

20. An antibody, which can specifically bind to the polypeptide according to claim 1.

21. A polynucleotide, which is represented by a nucleotide sequence having at least 20 sequential nucleotides in any of the sequences of SEQ ID NOS: 33 to 63 in the Sequence Listing, a sequence complementary to the nucleotide sequence, or a nucleotide sequence which is hybridizable with the nucleotide sequence under a stringent condition.

22. A polynucleotide, which is represented by a nucleotide sequence having at least 20 sequential nucleotides in any of the sequences of SEQ ID NOS: 43, 44, 61, and 62 in the Sequence Listing, a sequence complimentary to the nucleotide sequence, or a nucleotide sequence which is hybridizable with the nucleotide sequence under a stringent condition.

23. A polynucleotide, which is represented by a nucleotide sequence having at least 20 sequential nucleotides in any of the sequences of SEQ ID NOS: 87 to 93 in the Sequence Listing, a sequence complimentary to the nucleotide sequence, and a nucleotide sequence which is hybridizable with the nucleotide sequence under a stringent condition.

24. A probe, comprising a label and the polynucleotide according to claim 21.

25. A reagent for diagnosis, comprising the probe according to claim 24.

26. The reagent for diagnosis according to claim 25, which is used for a diagnosis of diabetes, a diabetic complication, Alzheimer's Disease, dialysis amyloidosis, a cancer, a periodontal disease, and an aging-related disease.
